SEQUENCE LISTING (18 sequences)

SEQ ID NO: 1 - protein, 407 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 2 - protein, 11 aa, Homo sapiens
RPKPQQFFGLM

SEQ ID NO: 3 - protein, 7 aa, Artificial Sequence
  SP variant
  AMIDATION (7)..(7)
QQFFGLM

SEQ ID NO: 4 - protein, 6 aa, Artificial Sequence
  SP variant
  AMIDATION (6)..(6)
QFFGLM

SEQ ID NO: 5 - protein, 5 aa, Artificial Sequence
  SP variant
  AMIDATION (5)..(5)
FFGLM

SEQ ID NO: 6 - protein, 4 aa, Artificial Sequence
  SP variant
  AMIDATION (4)..(4)
FGLM

SEQ ID NO: 7 - protein, 4 aa, Artificial Sequence
  IGF1 derivative free peptide
SSSR

SEQ ID NO: 8 - protein, 11 aa, Artificial Sequence
  SP fusion peptide
  AMIDATION (11)..(11)
SSSRQQFFGLM

SEQ ID NO: 9 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 10 - protein, 11 aa, Artificial Sequence
  Linker
GGSGGGSGGSG

SEQ ID NO: 11 - protein, 10 aa, Artificial Sequence
  Linker
GGSGGGSGSG

SEQ ID NO: 12 - protein, 8 aa, Artificial Sequence
  Linker
GGSGGGSG

SEQ ID NO: 13 - protein, 10 aa, Artificial Sequence
  Linker
GGGGSGGGGS

SEQ ID NO: 14 - protein, 8 aa, Artificial Sequence
  Linker
GGGSGGGS

SEQ ID NO: 15 - protein, 6 aa, Artificial Sequence
  Linker
GGSGGS

SEQ ID NO: 16 - DNA, 24 bp, Artificial Sequence
  NK1R F primer
ccaacacctccaccaacacttctg

SEQ ID NO: 17 - DNA, 23 bp, Artificial Sequence
  NK1R R1 primer
gccacagctgtcatggagtagat

SEQ ID NO: 18 - DNA, 20 bp, Artificial Sequence
  NK1R R2 primer
tccagactgccttgggaaaa

(19) United States
(12) Patent Application Publication
(10) Pub. No.: US 2018/0243363 A1
(43) Pub. Date: Aug. 30, 2018

(54) NEUROKININ 1 RECEPTOR AGONIST MEDIATED PROTECTION OF THE EYE

(71) Applicant: WAYNE STATE UNIVERSITY, Detroit, MI (US)

(72) Inventors: Susmit Suvas, Rochester, MI (US); Subhash Gaddipati, Ann Arbor, MI (US)

(73) Assignee: WAYNE STATE UNIVERSITY, Detroit, MI (US)

(21) Appl. No.: 15/979,320

(22) Filed: May 14, 2018

Related U.S. Application Data
(62) Division of application No. 15/445,817, filed on Feb. 28, 2017.
(60) Provisional application No. 62/301,389, filed on Feb. 29, 2016.

(51) Int. Cl. A61K 38/08; A61K 38/07; A61K 9/06; A61K 9/00; A61K 38/30

(52) U.S. Cl. CPC A61K 38/08; A61K 47/48215; A61K 38/07; A61K 38/30; A61K 9/06; A61K 9/0048

(57) ABSTRACT

Neurokinin 1 receptor (NK1R) agonist mediated protection of the eye is described. The protection can reduce dry eye and ocular infections, particularly in individuals with reduced NK1R activity.

### CROSS-REFERENCE TO RELATED APPLICATION

This application is a U.S. Divisional Patent Application based on U.S. patent application Ser. No. 15/445,817, filed on Feb. 28, 2017, which claims priority to U.S. Provisional Patent Application No. 62/301,389 filed Feb. 29, 2016, each of which is incorporated by reference herein in its entirely.

### STATEMENT OF GOVERNMENT INTEREST

This invention was made with government support under R01 EY022417 awarded by the National Institutes of Health. The government has certain rights in the invention.

### FIELD OF THE DISCLOSURE

The current disclosure provides neurokinin 1 receptor (NK1R) agonist mediated protection of the eye. The protection can reduce dry eye and ocular infections, particularly in individuals with reduced NK1R activity.

### REFERENCE TO SEQUENCE LISTING

A computer readable text file, entitled “Sequence Listing.txt” created on or about Feb. 27, 2017, with a file size of 6.74 KB, contains the sequence listing for this application and is incorporated by reference herein in its entirety.

### BACKGROUND OF THE DISCLOSURE

Neurokinin 1 Receptor (NK1R) antagonists can be used to treat a number of conditions, such as alcoholism, Sezary Syndrome (aggressive form of cutaneous T-cell lymphoma), epidermolysis Bullosa (rare diseases that cause the skin to blister), nausea, vomiting, post-traumatic stress disorder, and HIV infection. In fact, numerous clinical trials are underway around the globe for new treatments utilizing NK1R antagonists. Exemplary NK1R antagonists include Aprepitant, VPD-737, GSK1144814, Vestipitant, Casopitant (GW679769), and Orvepitant (GW823296).

NK1R regulates diabetic corneal epithelial wound healing (Yang et al., Diabetes. 2014; 63(12):4262-74). However, the role of NK1R in maintaining the homeostasis/integrity of the ocular surface in the absence of a wound has not been known.

### SUMMARY OF THE DISCLOSURE

The current disclosure provides that reduced Neurokinin 1 Receptor (NK1R) function destabilizes the ocular surface, leading to dry eye and increased ocular infections. Thus, the current disclosure provides administration of NK1R agonists to maintain or restore homeostasis/stability of the ocular surface, to treat dry eye, and/or to reduce ocular infections, particularly in individuals with suppressed or reduced NK1R activity. In particular embodiments, when an individual is receiving a systemic NK1R antagonist to treat a condition, the same individual can administer an NK1R agonist to the ocular surface to reduce destabilization that would otherwise occur. In this manner, the current disclosure provides maintenance and/or restoration of ocular surface integrity, and prevents or reduces the occurrence of dry eye and infections in these individuals.

### BRIEF DESCRIPTION OF THE FIGURES

Many of the drawings submitted herein are better understood in color, which is not available in patent application publications at the time of filing. Applicants consider the color versions of the drawings as part of the original submission and reserve the right to present color images of the drawings in later proceedings.

FIGS. 1A-1G. Lack of NK1R leads to alteration in corneal epithelial homeostasis. (1A) Slit lamp bio-microscope images of naïve WT (top) and NK1R^{-/-} (lower) showing corneal surface. (1B) Representative Hematoxylin and Eosin stained corneal tissue sections (8μ thick) from both groups at 20× magnification, boxed area shows the higher magnified view of that region. Apical corneal epithelial cell sloughing from the ocular surface of NK1R^{-/-} mice indicated by arrows. (1C) Graphical representation for the number of epithelial cells quantified from the H&E stained corneal section of VVT and NK1R^{-/-}. (1D) Representative corneal sections stained with anti BrdU Ab after 18 hours of BrdU pulsing in VVT and NK1R^{-/-} mice (BrdU positive cells indicated by arrows). (1E) Whole mount naïve corneas stained with E-Cadherin in VVT and NK1R^{-/-} mice (patchy staining labeled with star) (1F and 1G) Epithelial cells from limbal and central corneal regions of whole mount cornea stained with Zo1 in VVT and NK1R^{-/-} (Exfoliated cells or unstained patches indicated with star). P values were calculated using two-tail student's t-test (****p<0.0001 significant).

FIGS. 2A-2C. Lack of NK1R changes the homeostasis of corneal epithelial and limbal dendritic cells. (2A) Whole mount (montage images with 10×) corneas of naïve B6 and NK1R^{-/-} mice stained with FITC conjugated anti-CD11c antibody (HL3 Clone). Scatter plot shows the total number of CD11c positive cells/each cornea from both the groups. (2B) Low magnified limbal region of VVT and NK1R^{-/-} whole mount corneas stained with FITC conjugated anti-CD11c antibody (N418 clone, 5×). Dotted line denotes the limbal region. Scatter plot shows the total number of CD11c positive cells at limbus/cornea from both the groups. (2C) Co-localization of CD11c (N418) and CD11b in the limbal region of NK1R^{-/-} mice corneal whole mounts. Nucleus was stained with DAPI (20×). Data shown is the representative of three independent experiments. Cj-Conjunctiva, C Cornea-Central cornea. P values were calculated using two-tail student's t-test (**p<0.01 and *p<0.05 significant).

FIG. 3. Agarose gel electrophoresis of PCR products from NK1R^{-/-} mouse along with appropriate controls showing the 236 bps (NK1R^{-/-}) and 369 bps (intact receptor) amplicons. Primers used for genotyping are provided (SEQ ID NOs: 16-18).

FIG. 4. Confocal images of naïve corneal whole mounts stained with E-cadherin and DAPI (nuclear staining) showing hyperplastic crowded corneal wing cells in NK1R^{-/-} mice compared with B6 (20×).

FIG. 5. Naïve corneal whole mounts montage pictures of B6 and NK1R^{-/-} stained with CD11c antibody (N418 clone) showing more cells at limbal area of NK1R^{-/-} mice (5×).

FIGS. 6A-6C. Lack of NK1R alters the corneal nerve density, tear level but does not affect the corneal sensitivity. (6A) Confocal images of Tuj1 stained whole mount corneas from uninfected B6 and NK1R^{-/-} mice. Images are montages images acquired with 20× objective lens. Scatter plots denote nerve leash number and limbal stromal nerve trunk branch points from both groups of mice. (6B) Measurement of corneal sensitivity with a Cochet and Bonnet Esthesiometer. (6C) Image of phenol red threads used for measuring the tear quantity in B6 (top) and NK1R^{-/-} (bottom) mice. The extent of color change from the thread edge denotes the abundance of tears. Scatter plot represents the volume (in mm) of tear absorption by phenol red coated cotton thread from both the groups. Data shown is the representative of two independent experiments. P values were calculated using two-tail student's t-test (****p<0.0001, **p<0.01 and ns p>0.05).

FIGS. 7A-7E. Lack of NK1R promotes an early development of herpes stromal keratitis (HSK) and corneal angiogenesis. (7A) Representative slit lamp pictures of B6, NK1R^{-/-} mice eyes on day 11 post-ocular HSV1 infection. (7B) Frequency of eyes with severe HSK in both groups of mice. (7C and 7D) Scatter plots showing HSK and angiogenesis grading on day 9, 11, 14 and 16-post ocular infection. (7E) Cytokine and chemokine array blots of B6 and NK1R^{-/-} mice from the corneal lysates prepared on day 9 post-ocular infection. Quantitation of three important molecules involved in regulating HSK and angiogenesis development is represented as a bar graph. Data shown is the representative of three independent experiments. P values were calculated using two-tail student's t-test (****<0.0001, ***<0.001**p<0.01, *p<0.05 significant and p>0.05 non-significant).

FIGS. 8A, 8B. Increased influx of CD11c expressing cells into inflamed corneas and draining lymph nodes of NK1R^{-/-} mice after ocular HSV-1 infection. (8A) Corneal whole mounts stained with FITC conjugated anti-CD11c (N418) antibody from B6 and NK1R^{-/-} mice on day 2 post-ocular HSV-1 infection. More magnified limbal, paracentral and central corneal images for both groups are included. (8B) Representative FACS plots denote the number of CD11c+ cells in DLN of infected mice on day 3 and day 5 post-infection. Scatter plots demonstrate the number of CD11c+ cells in DLNs of individual mice from both groups. Data shown is the representative of two independent experiments. P values were calculated using two-tail student's t-test (*p<0.05 significant and p>0.05 non-significant).

FIG. 9A, 9B. Increased expansion and Th1 differentiation of CD4 T cells in DLNs of HSV-1 infected NK1R^{-/-} mice. (9A) Representative FACS plots showing frequencies of Ki67 expressing CD4 T cells in the DLN of B6 and NK1R^{-/-} mice on day 3 post-ocular HSV-1 infection. Scatter plot shows absolute numbers of CD4+Ki67+ cells in DLNs of uninfected and HSV-1 infected mice from both groups on day 3 and 5 post-infection. (9B) Representative FACS plots denoting IFN-γ producing CD4 T cells after ex-vivo re-stimulation of lymph node cells from both groups of mice with UV inactivated HSV-1 on Day 7 post-infection. Scatter plot demonstrates absolute numbers of IFN-γ producing CD4 T cells from both groups of mice. (****<0.0001, *p<0.05 significant and p>0.05 non-significant).

FIG. 10. Increased influx of CD4 T cells in infected corneas of NK1R^{-/-} mice. (A) Representative FACS plots of CD4 T cells in infected corneas from both groups of mice on day 11 and 17 post-infection. Scatter plots demonstrate the absolute numbers and frequencies of CD4 T cells in individual corneal tissue from both the groups on day 11 and day 17 post-infection. Data shown is the representative of two independent experiments. P values were calculated using two-tail student's t-test. (***p<0.001, *p<0.05 significant).

FIG. 11. Normal corneal whole mounts stained for CD31 showing dilated limbal feeder blood vessels in NK1R^{-/-} compared with B6 limbal blood vessels (20×).

FIGS. 12A, 12B. Increased tear volume at D7 and D10 post ocular HSV-1 infection (POI) in GR73632 I.P treated eyes compared to the control treated group. Phenol Red thread (yellow color) images showing the thread wetness (red color) measured in mm. Significant increase in tear volume is shown at D7 POI in control vs GR73632 treated group with corresponding thread images of both groups (*p<0.05) in the lower left panel (12A). Significant increase in tear volume is shown at D10 POI in control vs GR73632 treated group with corresponding thread images in both groups (**p<0.01) in the lower right panel (12B).

FIG. 13. Development of lymphangiogenesis in NK1R^{-/-} mice. NK1R^{-/-} mice developed corneal lymphatics. Wholemounts of naïve Corneas stained with Lyve1 demonstrating more lymphatic area in NK1R^{-/-} mice.

FIG. 14. Alteration in Tear Film after NK1R antagonist administration. Treatment with the systemic NK1R antagonist Spantide I reduces the volume of basal tears in C57Bl/6 mice.

FIG. 15. Schematic representation of corneal epithelial and dendritic cell homeostasis in B6 and NK1R^{-/-} mice. NK1R^{-/-} mice corneal epithelium is hyperplastic with reduced peripheral nerve leashes, sub-basal nerve trunk branch points and dendritic shaped corneal DC's but significantly increased limbal DC's compared with B6 cornea. HSV-1 infection leads to mobilization of increased number of limbal DCs into inflamed cornea and then to the DLN of NK1R^{-/-} mice resulting in an increased expansion and Th1 differentiation of CD4 T cells. Increased influx of CD4 T cells into infected eyes causes an early development of HSK in NK1R^{-/-} mice.

FIG. 16 depicts anatomy of aspects of the eye.

FIG. 17 depicts glycocalyx and the tear film. The glycocalyx is a glycoprotein-polysaccharide covering that surrounds the cell membranes of some bacteria, epithelia and other cells. In the eye, GlycoCalyx are long chain molecules that help hold mucin to the corneal surface. Formed by corneal cells, glycocalyx migrate out from the surface of the corneal microvilli to form a hydrophilic network that holds mucin on the ocular surface. Holding mucin to the ocular surface creates a water attraction, as well as protection against bacterial pathogens. Since the corneal surface is naturally water repellent, damage to glycocalyx and corneal surface cells may be caused by insufficient mucin. This can cause the tear film to destabilize and break up before a blink can occur, exposing the injured cornea to the air and bacterial pathogens.

FIG. 18. Additional exemplary supporting sequences.

### DETAILED DESCRIPTION

Neurokinin 1 Receptor (NK1R) antagonists can be used to treat a number of conditions, such as alcoholism, Sezary Syndrome (aggressive form of cutaneous T-cell lymphoma), epidermolysis Bullosa (rare diseases that cause the skin to blister), nausea, vomiting, post-traumatic stress disorder, and HIV infection. In fact, numerous clinical trials are underway around the globe for new treatments utilizing NK1R antagonists. Exemplary NK1R antagonists include Aprepitant, VPD-737, GSK1144814, Vestipitant, Casopitant (GW679769), and Orvepitant (GW823296).

NK1R regulates diabetic corneal epithelial wound healing (Yang et al., Diabetes. 2014; 63(12):4262-74). However, the role of NK1R in maintaining the homeostasis/integrity of the ocular surface in the absence of a wound has not been known.

The current disclosure provides that reduced NK1R function destabilizes the ocular surface, leading to dry eye and increased ocular infections. Thus, the current disclosure provides administration of NK1R agonists to maintain and/or restore homeostasis/stability of the ocular surface, to treat dry eye, and/or to reduce ocular infections, particularly in individuals with suppressed or reduced NK1R activity. In particular embodiments, when an individual is receiving an NK1R antagonist to treat a condition, the same individual can administer an NK1R agonist to the ocular surface to reduce destabilization that would otherwise occur. In this manner, the current disclosure provides maintenance or restoration of ocular surface homeostasis/integrity, and prevents or reduces the occurrence of dry eye and infections in these individuals.

Dry eye refers to the condition in which there are insufficient tears to lubricate, nourish, and maintain the health of the front surface of the eye. The reduced tear film associated with dry eye reduces the ability to wash away foreign particles to keep the surface of the eye smooth and clear. Dry eye may also increase the risk of ocular infections.

People with dry eyes either do not produce enough tears or have a poor quality of tears. Dry eye can lead to damage to the glycocalyx (causing damage to the cilia and causing the cells to detach from the basal surface), a decrease in mucin, and turbidity in the tear film on the eye. Dry eye is a common and often chronic problem, particularly in older adults.

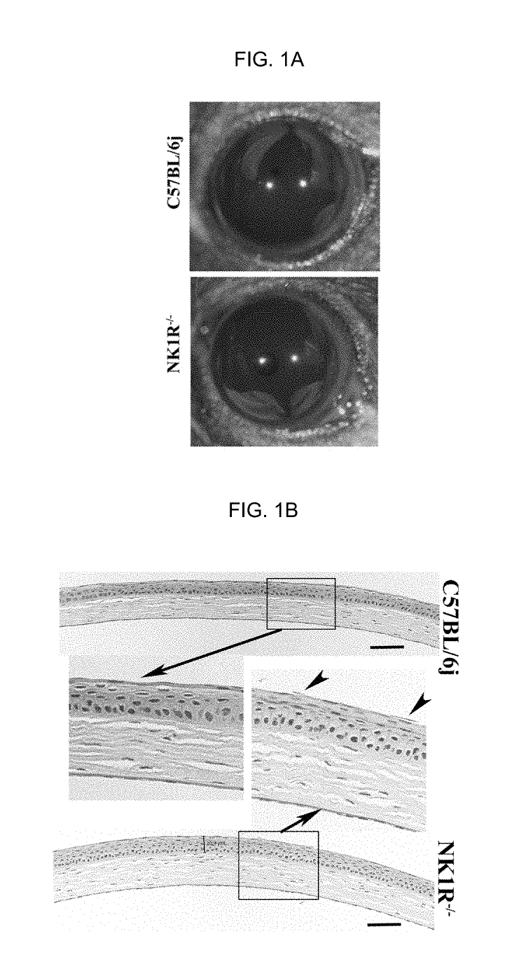

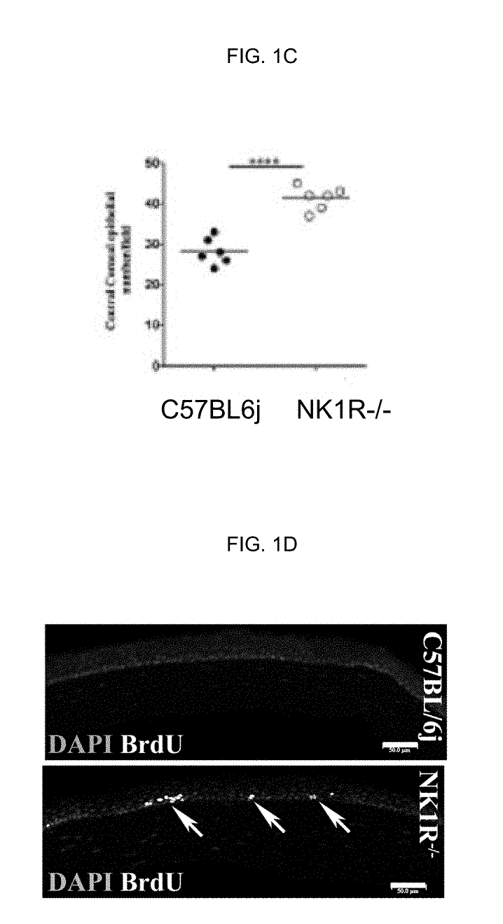

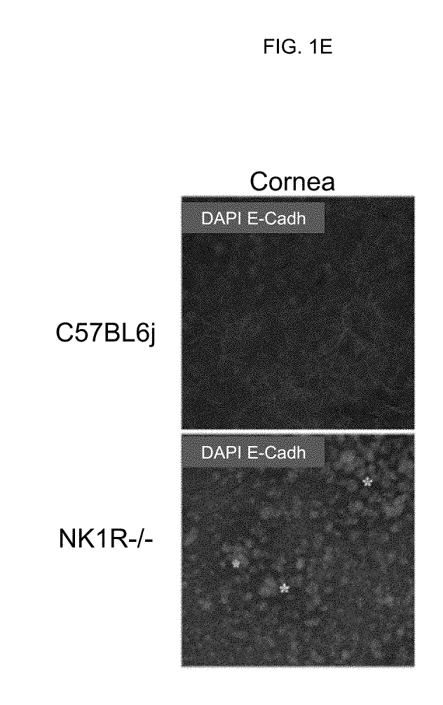

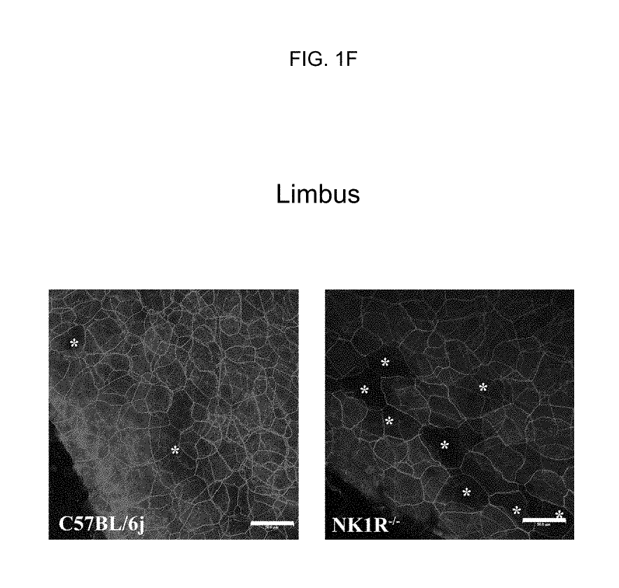

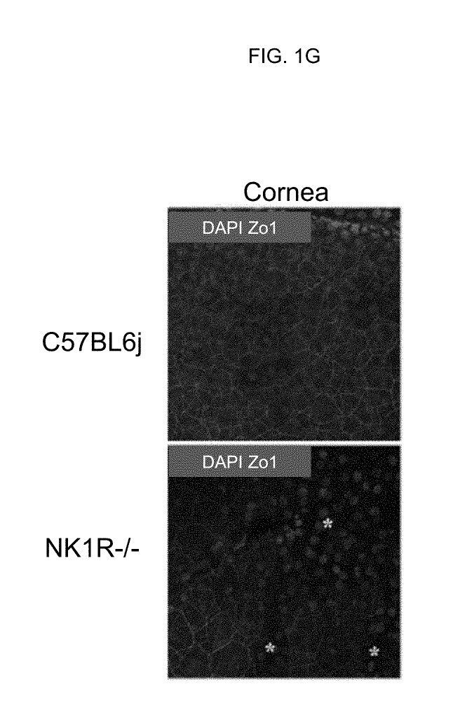

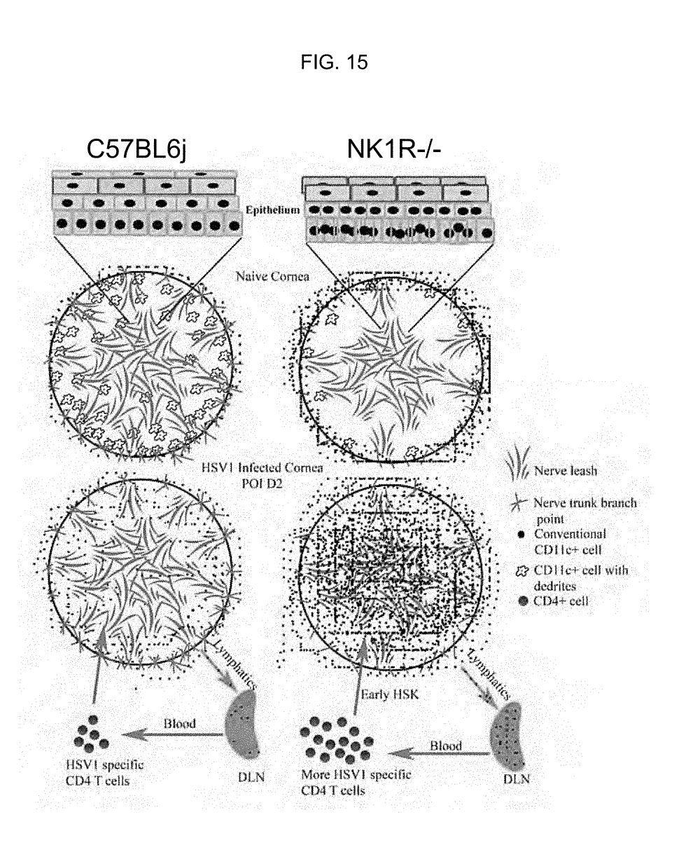

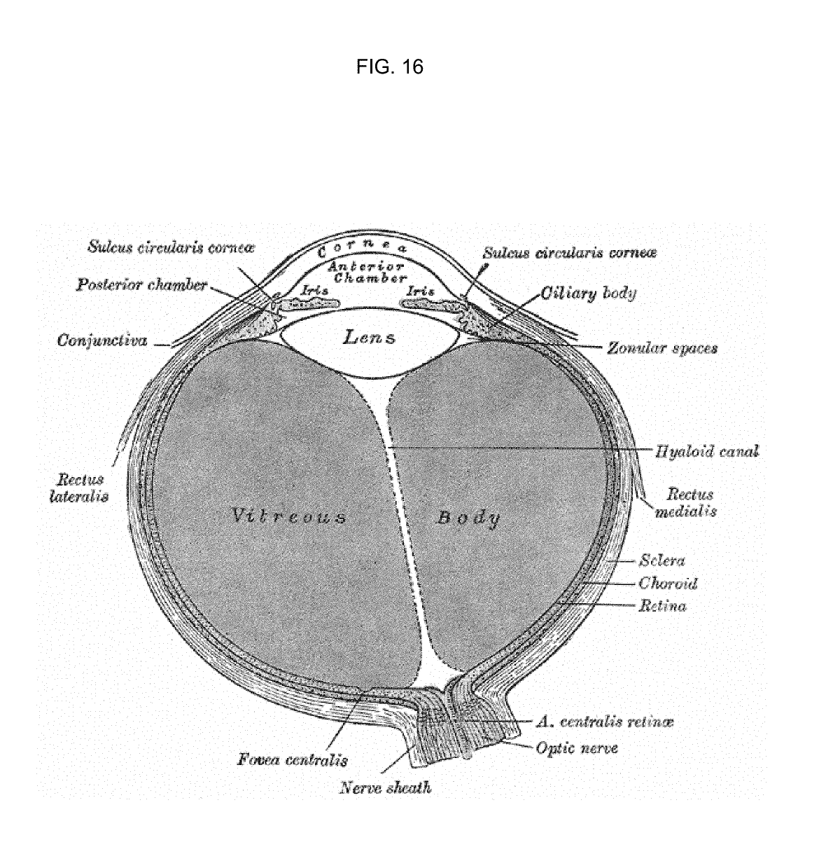

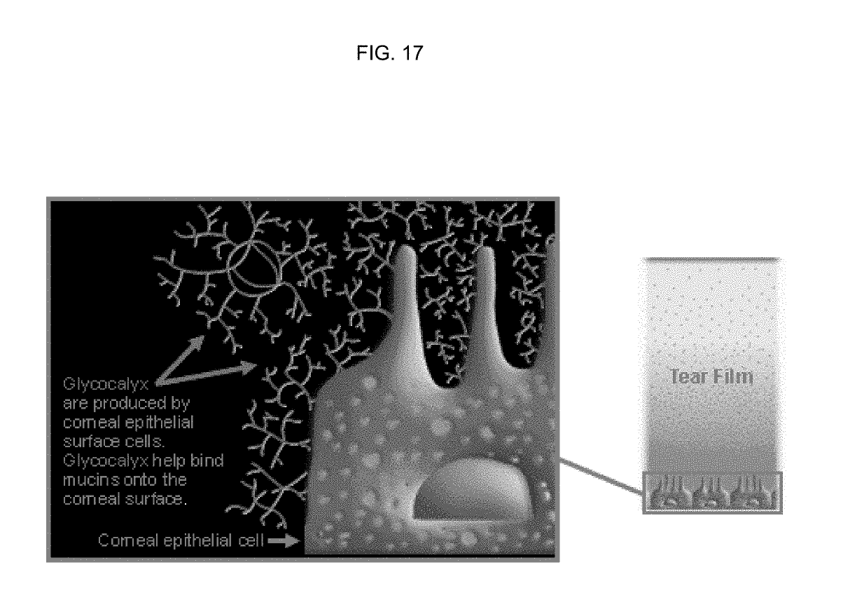

That reduced NK1R function destabilizes the ocular surface, leading to dry eye can be seen in FIGS. 1A-1G. More particularly, there are noticeable differences in the conjunctiva and cornea demonstrating development of the dry eye condition. The conjunctiva lines the inside of the eyelids and covers the sclera (white part of the eye). It helps lubricate the eye by producing mucus and tears, although a smaller volume of tears than the lacrimal gland. It also contributes to immune surveillance and helps to prevent the entrance of microbes into the eye. See FIG. 15. The conjunctiva is composed of non-keratinized, stratified columnar epithelium with goblet cells, and also stratified columnar epithelium. Goblet cells are glandular, modified simple columnar epithelial cells whose function is to secrete gel-forming mucins, the major components of mucus. The goblet cells mainly use the merocrine method of secretion, secreting vesicles into a duct, but may use apocrine methods, budding off their secretions, when under stress.

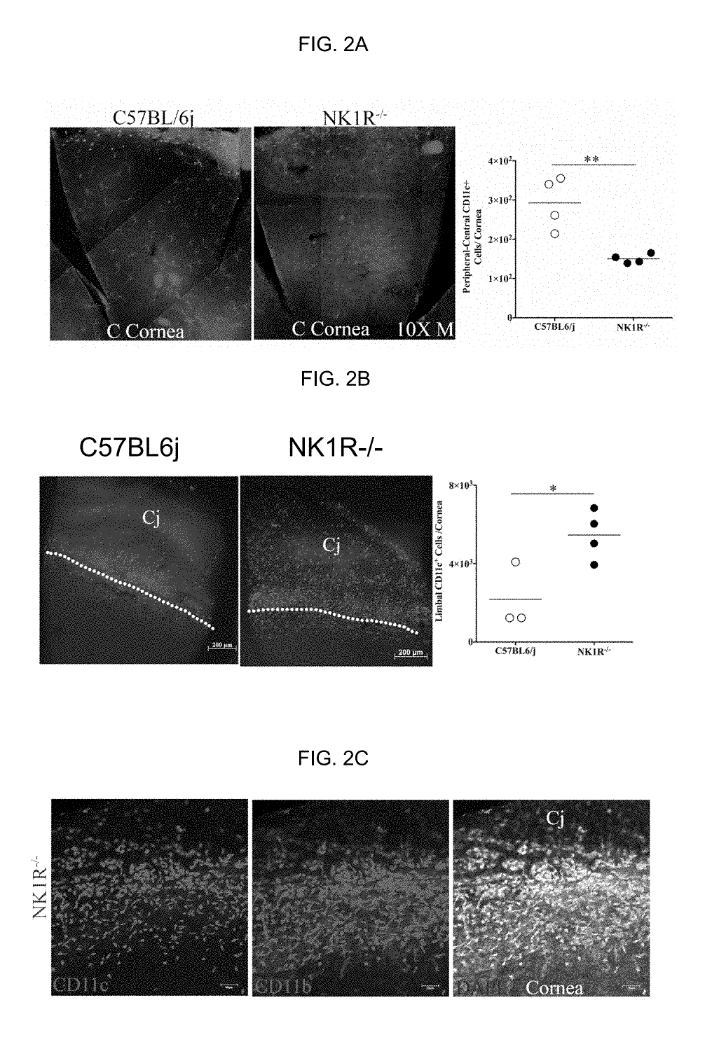

As disclosed herein, the development of clinical features of dry eye disease were evidenced by corneal epitheliopathy, corneal lymphangiogenesis (development of lymphatic vessels in the cornea), and corneal neuropathy, each detected in subjects with reduced NK1R activity. For example, the current disclosure shows evidence of epithelial cell separation from the surface of the cornea and a decrease in cadherin's and tight junction proteins. Increased corneal epithelial cell density, decreased corneal epithelial cell size and increased corneal epithelial cell turnover were noted in subjects with reduced NK1R activity. Reduced numbers of corneal epithelial dendritic cell were also noted with reduced NK1R activity. Development of lymphatic vessels in cornea, the hallmark of dry eye was also noted in subjects with reduced NK1R activity. There is also a decrease in corneal neurons and tear formation. Further, the conjunctiva of the eye was inflamed with dilation of blood vessels. There was also a dramatic increase of immune cells in the conjunctival and near the limbal region of cornea as determined by staining for CD11c and CD11b (see FIGS. 2A-2C). CD11c is a type I transmembrane protein found at high levels on most human dendritic cells, but also on monocytes, macrophages, neutrophils, and some B cells that induces cellular activation and helps trigger neutrophil respiratory burst. The limbal region (also known as the corneal limbus) is the border of the cornea and the sclera. The limbus is a common site for the occurrence of corneal epithelial neoplasm. The limbus contains radially-oriented fibrovascular ridges known as the palisades of Vogt that may harbour a stem cell population.

In particular embodiments, the current disclosure provides administration of one or more NK1R agonists to restore homeostasis/stability of the ocular surface, to treat dry eye, and/or to reduce ocular infections in individuals with suppressed or reduced NK1R activity. In particular embodiments, the current disclosure includes treating mild dry eye developed due to different causes that are not mutually exclusive such as age-related changes, diabetes, goblet cell loss, and/or systemic NK1R antagonist use. In particular embodiments, the current disclosure treats the cause of dry eye disease through conditioning the ocular surface by increased tear production, restoring the surface cellular glycocalyx and/or stabilizing differentiated cellular adhesion and tight junction proteins. In particular embodiments, the disclosed treatments help control immune cell infiltration into the ocular surface especially to conjunctival tissue and the limbal regions of cornea.

In particular embodiments, the current disclosure provides administration of NK1R agonists to maintain or restore homeostasis between corneal epithelial cells and corneal epithelial dendritic cells. In particular embodiments, the current disclosure provides administration of NK1R agonists to maintain or restore homeostasis between corneal epithelial cells and conjunctival dendritic cells. In particular embodiments, the current disclosure provides administration of NK1R agonists to maintain or restore regularity of the corneal surface. In particular embodiments, the current disclosure provides administration of NK1R agonists to maintain or restore tear level. In particular embodiments, the current disclosure provides administration of NK1R agonists to reduce apical corneal epithelial cell sloughing. In particular embodiments, the current disclosure provides administration of NK1R agonists to prevent or reduce corneal stromal edema. In particular embodiments, the current disclosure provides administration of NK1R agonists to prevent or reduce corneal epithelial hyperplasia. In particular embodiments, the current disclosure provides administration of NK1R agonists to prevent or reduce ocular infection. In particular embodiments, the current disclosure provides administration of NK1R agonists to prevent or reduce changes in corneal opacity, corneal neuropathy, and superficial punctate keratitis (SPKs).

Particular aspects of the disclosure are now described in more detail.

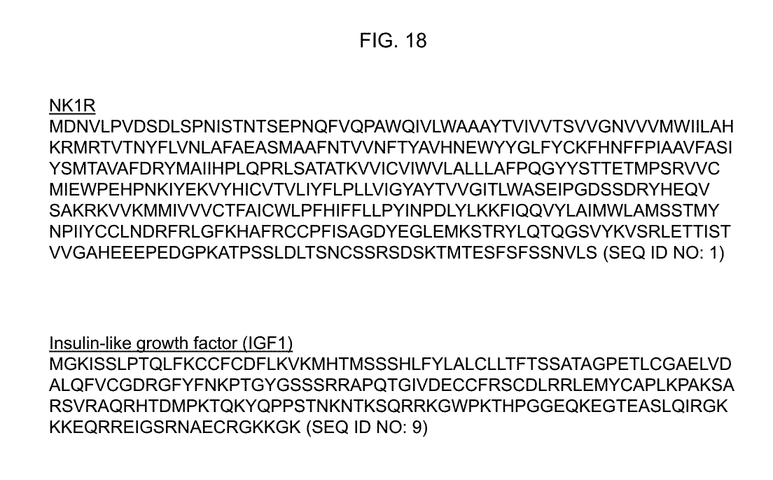

The NK1 Receptor (NK1R). There are three known mammalian tachykinin receptors termed NK1, NK2 and NK3. All are members of the 7 transmembrane G-protein coupled receptor family and induce the activation of phospholipase C, producing inositol triphosphate. The receptor is found in the central nervous system and peripheral nervous system. The endogenous ligand for this receptor is Substance P (SP), although it has some affinity for other tachykinins. The binding of SP to NK1R is associated with the transmission of stress signals and pain, the contraction of smooth muscles, and inflammation. SP has also recently been found in the glococalyx of the eye, a primary target of the current disclosure. The NK1R pathway may be involved in instances of dry eye due to age, diabetes and/or goblet cell loss. An exemplary sequence representing the NK1R is provided in SEQ ID NO: 1, see FIG. 18).

NK1R Agonists. Substance P (SP) is the primary natural agonist of NK1R. SP is a peptide composed of a chain of 11 amino acid residues (RPKPQQFFGLM; SEQ ID NO: 2). It is ubiquitous and considered a neuropeptide, acting as a neurotransmitter and as a neuromodulator. This peptide binds to NK1R with high affinity and is involved in vasodialation, inflammation, pain, mood, anxiety, learning, emesis, cell growth, proliferation, angiogenesis and migration.

Variants, analogs, and derivatives of NK1R agonists can also be used within the teachings of the current disclosure, so long as the variant, analog or derivative of the NK1R agonist retains the biological effect of the reference molecule (e.g., SP or GR73632). “Retains the biological effect” means there is not a statistically significant difference in the effectiveness of the variant, analog or derivative, or other NK1R agonist and the reference peptide when utilized in a research model of dry eye disclosed herein. Exemplary SP variants (which can also include SP-derived peptides and/or SP derivatives and/or analogs) include QQFFGLM-NH₂ (SEQ ID NO: 3), QFFGLM-NH₂ (SEQ ID NO: 4), FFGLM-NH₂ (SEQ ID NO: 5), and FGLM-NH₂ (SEQ ID NO: 6), wherein —NH₂ represents C-terminal amidation.

GR73632 is another example of an NK1R agonist. GR73632 has the structure:

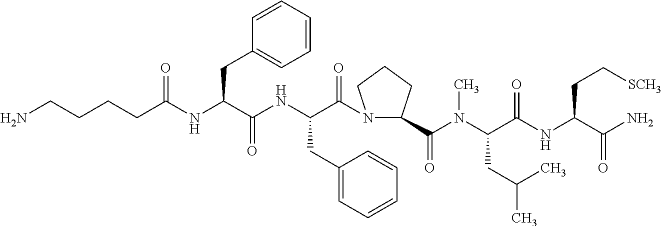 [SMILES: CSCC[C@H](NC(=O)[C@H](CC(C)C)N(C)C(=O)[C@@H]1CCCN1C(=O)[C@H](Cc1ccccc1)NC(=O)[C@H](Cc1ccccc1)NC(=O)CCCCN)C(N)=O]

GR64349 is another example of an NK1R agonist. GR64349 has the structure:

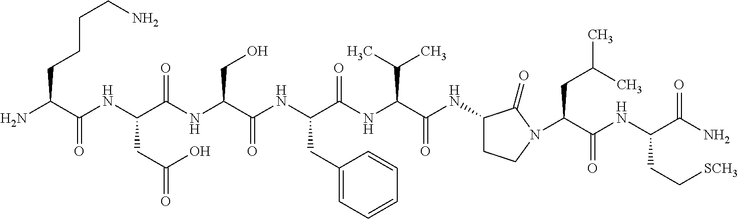 [SMILES: CSCC[C@H](NC(=O)[C@H](CC(C)C)N1CC[C@H](NC(=O)[C@@H](NC(=O)[C@H](Cc2ccccc2)NC(=O)[C@H](CO)NC(=O)[C@H](CC(=O)O)NC(=O)[C@@H](N)CCCCN)C(C)C)C1=O)C(N)=O]

In particular embodiments, an NK1R agonist is used in combination with an Insulin-like growth factor 1 (IGF1) protein or peptide. In particular embodiments, an IGF1 protein or an IGF1-derived peptide, in combination with an NK1R agonist, can increase the biological activity and half-life on an ocular surface, contribute to maintenance of ocular surface integrity, and/or contribute to prevention or treatment of dry eye. An exemplary IGF1 sequence is SEQ ID NO: 9 (UniProt ID P05019, see FIG. 18), and an exemplary peptide sequence derived from IGF1 is SSSR, SEQ ID NO: 7). Thus, as used herein, IGF1 protein includes the full protein sequence of IGF1 whereas an IGF1 peptide is a fragment of the IGF1 protein. IGF1 fragments can consist of SEQ ID NO: 7 or can comprise SEQ ID NO: 7, such as by including 1-20 amino acids adjacent to SEQ ID NO: 7 in the 3′ and/or 5′ prime direction.

In particular embodiments the NK1R agonist is a fusion protein or peptide containing an SP or SP variant, and an IGF1 protein or peptide motif. An exemplary SP-IGF1 fusion peptide is SSSRQQFFGLM-NH₂ (SEQ ID NO: 8). A fusion protein or peptide containing an NK1R agonist domain and an IGF1 domain may include a linker. A linker is an amino acid sequence which can provide flexibility and room for conformational movement between protein or peptide domains. Any appropriate linker may be used. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target. Commonly used flexible linkers include Gly-Ser linkers such as GGSGGGSGGSG (SEQ ID NO: 10), GGSGGGSGSG (SEQ ID NO: 11) and GGSGGGSG (SEQ ID NO: 12). Additional examples include: GGGGSGGGGS (SEQ ID NO: 13); GGGSGGGS (SEQ ID NO: 14); and GGSGGS (SEQ ID NO: 15). Linkers that include one or more antibody hinge regions and/or immunoglobulin heavy chain constant regions, such as CH3 alone or a CH2CH3 sequence can also be used.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues.

Fusion proteins can be formed using any appropriate recombinant expression technique known to those of ordinary skill in the art.

In particular embodiments, the NK1R agonists can be conjugated to polyethylene glycol (PEG), which refers to a polymer of repeating ethylene glycol units. PEGylation of a protein or peptide can be performed by reacting the protein or peptide with a PEG molecule that contains a functional group (e.g., an NHS ester group) that is reactive with a particular moiety present in a peptide or protein (e.g., the N-terminus). In particular embodiments, SEQ ID NO: 8 and SEQ ID NO: 3 are PEGylated to form PEG-SSSRQQFFGLM-NH₂ and PEG-N-QQFFGLM-NH₂, respectively. In particular embodiments, PEGylation of NK1R agonists can increase retention time on an ocular surface without compromising biological activity. Several sizes of PEGs are commercially available (Nektar Advanced PEGylation Catalog 2005-2006; and NOF DDS Catalogue Ver 7.1).

“Variants” of proteins disclosed herein include proteins having one or more amino acid additions, deletions, stop positions, or substitutions, as compared to a protein disclosed herein.

An amino acid substitution can be a conservative or a non-conservative substitution. Variants of proteins disclosed herein can include those having one or more conservative amino acid substitutions. A “conservative substitution” involves a substitution found in one of the following conservative substitutions groups: Group 1: alanine (Ala or A), glycine (Gly or G), Ser, Thr; Group 2: aspartic acid (Asp or D), Glu; Group 3: asparagine (Asn or N), glutamine (Gln or Q); Group 4: Arg, lysine (Lys or K), histidine (His or H); Group 5: Ile, leucine (Leu or L), methionine (Met or M), valine (Val or V); and Group 6: Phe, Tyr, Trp.

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

In particular embodiments, variants include 1 or 2 conservative amino acid substitutions.

“Analog” (also “structural analog” or “chemical analog”) refers to a compound that is structurally similar to another compound but differs with respect to a certain component, such as an atom, a functional group, or a substructure.

“Derivative” refers to a compound that is obtained from a similar compound or a precursor compound by a chemical reaction.

NK1R agonists alone or in combination with an IGF1 protein or peptide (“active ingredients”) can be formulated into compositions for administration to a subject. Salts and/or prodrugs of active ingredients can also be used. In particular embodiments an NK1R agonist and an IGF1 protein or peptide can be formulated into a composition together. In particular embodiments an NK1R agonist and IGF1 protein or peptide can each be formulated into separate compositions for administration.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of a protein or by hydrolysis of a biologically labile group.

Ophthalmic formulations can be prepared as solutions, suspensions, ointments, gels, emulsions, oils, and other dosage forms for topical administration. Aqueous solutions are generally preferred, based on ease of formulation, as well as a patient's ability to easily administer such compositions by means of instilling one to two drops of the solutions in the affected eyes. However, the compositions can also be suspensions (e.g., eye drops), viscous or semi-viscous gels, or other types of solid or semisolid compositions (e.g., ointments) or sustained release devices or mechanisms that are placed in or around the eye. In particular embodiments, aqueous formulations typically can be more than 50%, more than 75%, or more than 90% by weight water. In particular embodiments, an ointment is an oil-based or oil-and-water-based semi-solid or viscous formulation with a melting or softening point near body temperature. In particular embodiments, eye drops are a liquid formulation that can be applied to the eye as droplets. In particular embodiments, a gel is a jelly-like viscous formulation that includes a matrix of interacting molecules that confer viscosity, and a liquid (e.g., suspension) that is dispersed within the matrix.

Ophthalmic formulations can also be provided with tear substitutes. “Tear substitutes” refer to molecules or compositions which lubricate or “wet,” approximate the consistency of endogenous tears, aid in natural tear build-up, or otherwise provide temporary relief of eyes upon ocular administration. A variety of tear substitutes are known in the art and include: monomeric polyols, such as, glycerol, propylene glycol, and ethylene glycol; polymeric polyols such as polyethylene glycol; cellulose esters such hydroxypropylmethyl cellulose, carboxymethyl cellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include cellulose esters such as Bion® Tears (Alcon Research, Ltd., Fort Worth, Tex., U.S.A.), Celluvisc® (Allergan, Inc., Irvine, Calif., U.S.A.), Genteal® (Novartis Pharmaceuticals Corporation, East Hanover, N.J., U.S.A.), OccuCoat® (Barnes-Hind, Inc. Clearwater, Fla., U.S.A.), Refresh® (Allergan, Inc., Irvine, Calif., U.S.A.), Systane® (Novartis AG, Basel, Switzerland), Systane Ultra® (Novartis AG, Basel, Switzerland), Refresh Endura® (Allergan, Inc., Irvine, Calif., U.S.A.), Refresh Liquigel® (Allergan, Inc., Irvine, Calif., U.S.A.), Teargen II™ (McKesson), Tears Naturale® (Alcon (Puerto Rico), Inc., Humacao, Puerto Rico), Tears Naturale® II (Alcon (Puerto Rico), Inc., Humacao, Puerto Rico), Tears Naturale Free® (Alcon (Puerto Rico), Inc., Fort Worth, Tex., U.S.A.), and TheraTears® (Advanced Vision Research, Inc., Ann Arbor, Mich., U.S.A.); and polyvinyl alcohols such as Akwa Tears® (Akorn, Inc., Lake Forest, Ill., U.S.A.), HypoTears® (Novartis Pharmaceuticals Corporation, East Hanover, N.J., U.S.A.), Moisture Eyes® (Bausch & Lomb Incorporated, Rochester, N.Y., U.S.A.), Murine Tears® (Medtech Products, Inc., Irvington, N.Y., U.S.A.), Visine Tears® (Johnson & Johnson, New Brunswick, N.J. U.S.A.), and Soothe® (Bausch & Lomb Incorporated, Rochester, N.Y., U.S.A.). Tear substitutes may also include paraffins, such as the commercially available Lacri-Lube® (Allergan, Inc., Irvine, Calif., U.S.A.) ointments. Other commercially available ointments that are used as tear substitutes include Lubrifresh PM™ (Bausch & Lomb Incorporated), Moisture Eyes® PM (Bausch & Lomb Incorporated, Rochester, N.Y., U.S.A.) and Refresh P.M.® (Allergan, Inc., Irvine, Calif., U.S.A.).

Additional potential excipients for ophthalmic formulations include solubilizing agents, stabilizing agents, surfactants, demulcents, viscosity agents, diluents, inert carriers, preservatives, binders, and/or disintegrants. Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN® (Tween-Croda Americas, LLC, Wilmington, Del., PLURONICS® (Pluronic-BASF Corp., Mount Olive, N.J.), or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

Methods of Use. Methods disclosed herein include treating subjects (humans, veterinary animals, dogs, cats, reptiles, birds, etc.), livestock (horses, cattle, goats, pigs, chickens, etc.), and research animals (monkeys, rats, mice, fish, etc.) with active ingredients and/or salts and/or prodrugs thereof. Treating subjects includes delivering therapeutically effective amounts. Therapeutically effective amounts include those that provide effective amounts, prophylactic treatments, and/or therapeutic treatments.

An “effective amount” is the amount of an active ingredient necessary to result in a desired physiological change in a subject. Effective amounts are often administered for research purposes. Effective amounts disclosed herein maintain or restore ocular homeostasis and/or integrity in an animal or clinical research model such as those disclosed herein.

A “prophylactic treatment” includes a treatment administered to a subject who does not display signs or symptoms of ocular surface destabilization; or only displays early signs or symptoms of ocular surface destabilization such that treatment is administered for the purpose of promoting maintenance of ocular surface stability, reducing or preventing the development of dry eye and/or ocular infections. Thus, a prophylactic treatment functions as a preventative treatment against ocular surface destabilization, dry eye, and/or ocular infections.

A “therapeutic treatment” includes a treatment administered to a subject who displays symptoms or signs of ocular surface destabilization and is administered to the subject for the purpose of reducing or reversing the ocular surface destabilization.

As used herein, ocular surface destabilization is an effect disclosed herein that results in dry eye and/or increased ocular infections.

Maintaining homeostasis refers to maintaining, restoring, or improving (i) the balance of corneal epithelial cells to limbic dendritic cells; (ii) the balance of corneal epithelial cells to conjunctival dendritic cells; and/or (iii) the density of corneal epithelial dendritic cells, all in reference to a healthy eye.

Maintaining integrity of the ocular surface refers to maintaining, restoring, or improving (i) the tear level and/or reducing one or more of (i) apical corneal epithelial cell sloughing; (ii) changes in opacity; (iii) changes in angiogenesis; and/or (iv) occurrence of corneal stromal edema, all in reference to a healthy eye.

Effective therapeutically effective amounts can be evidenced by one or more of: subjective or objective feedback from a subject reporting alleviation of symptoms of dry eye or ocular infection; subjective or objective feedback from a doctor reporting alleviation of a physical manifestation of dry eye, ocular infection, altered homeostasis and/or reduced ocular integrity. In particular embodiments the objective feedback can be based on scoring corneal opacity on a scale of 0-5 or based on slit lamp examination.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by a physician, veterinarian, or researcher taking into account parameters such as physical and physiological factors including target area; severity of condition; degree of reduced NK1R function (based, for example, on dose of NK1R antagonist to treat a concurrent condition); prospective conditions; previous or concurrent therapeutic interventions; and idiopathy of the subject.

Useful dosing schedules can often range from 1-3 administration doses (e.g., eye drops) every 30 minutes; once an hour; once every 2 hours; once every 4 hours; once every 8 hours; once every 16 hours; once every 24 hours; once every 48 hours; or once a week. The timing of administration can vary from subject to subject, depending upon factors such as the severity of a subject's symptoms and the stage and type of condition.

Exemplary doses can include 0.0001 mg to 100 mg/administration dose of an active ingredient (e.g., an NK1R agonist and/or an IGF1 protein or peptide disclosed herein). The total daily dose can be 0.1 mg to 50.0 mg of the active ingredient administered to a subject, for example, one to three times a day. Additional useful doses can often range from 0.1 to 5 μg or from 0.5 to 1 μg/administration dose. In other examples, an administration dose can include, for example, 1 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 150 μg, 200 μg, 250 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 0.1 to 5 mg or from 0.5 to 1 mg active ingredient.

### Exemplary Embodiments

1. A method of protecting the eye in a subject in need thereof including administering a therapeutically effective amount of a Neurokinin 1 Receptor (NK1R) agonist to the subject thereby protecting the eye in the subject in need thereof.
2. A method of embodiment 1 wherein the subject is in need thereof due to reduced NK1R activity.
3. A method of embodiment 2 wherein the subject has reduced NK1R activity due to administration of an NK1R antagonist.
4. A method of any of embodiments 1-3 wherein the administering includes topical ocular administering.
5. A method of any of embodiments 1-4 wherein the protecting maintains, restores, or improves (i) the balance of corneal epithelial cells to limbal dendritic cells; (ii) the balance of corneal epithelial cells to conjunctival dendritic cells; and/or (iii) the density of corneal epithelial dendritic cells, all in reference to a healthy eye.
6. A method of any of embodiments 1-4 wherein the protecting (i) maintains, restores, or improves the tear level and/or (ii) reduces one or more of (a) apical corneal epithelial cell sloughing; (b) changes in opacity; (c) changes in lymphangiogenesis; and/or (d) occurrence of corneal stromal edema, all in reference to a healthy eye.
7. A method of any of embodiments 1-6 wherein the NK1R agonist includes Substance P or a Substance P variant.
8. A method of embodiment 7 wherein the NK1R agonist is selected from any of SEQ ID NOs: 2-6.
9. A method of any of embodiment 1-8 wherein the NK1R agonist is administered with IGF1 protein and/or an IGF1 peptide (in particular embodiments, formulated within a composition and/or as a fusion protein).
10. A method of embodiment 9 wherein the fusion protein includes SEQ ID NO: 8.
11. A method of any of embodiments 1-10 wherein the NK1R agonist is PEGylated.
12. A method of any of embodiments 1-11 wherein the NK1R agonist includes GR73632 or GR64349.
13. A method of any of embodiments 1-12 further including administering a therapeutically effective amount of an IGF1 protein or peptide.
14. A method of reducing dry eye and ocular infections in a subject in need thereof including administering a therapeutically effective amount of a Neurokinin 1 Receptor (NK1R) agonist to the subject thereby reducing dry eye and ocular infections in the subject in need thereof.
15. A method of embodiment 14 wherein the subject is in need thereof due to reduced NK1R activity.
16. A method of embodiment 14 or 15 wherein the subject has reduced NK1R activity due to administration of an NK1R antagonist.
17. A method of embodiment 16 wherein the administration of the NK1R antagonist is to treat a condition.
18. A method of embodiment 17 wherein the condition is alcoholism, Sezary Syndrome, epidermolysis Bullosa, nausea, vomiting, post-traumatic stress disorder, or HIV infection
19. A method of any of embodiments 14-18 wherein the administering includes topical ocular administering.
20. A method of any of embodiments 14-19 wherein the NK1R agonist includes Substance P, a Substance P variant, GR73632, or GR64349.
21. A method of embodiment 20 wherein the Substance P variant includes any of SEQ ID NOs: 3-6.
22. A method of any of embodiments 14-20 further including administering a therapeutically effective amount of an IGF1 protein or peptide.
23. A method of any of embodiment 21 wherein the NK1R agonist and IGF1 protein are formulated within a composition and/or as a fusion protein (e.g., SEQ ID NO: 8).
24. A topical ophthalmic formulation including an NK1R agonist.
25. A topical ophthalmic formulation of embodiment 24 formulated as eye drops, an ophthalmic gel or an ointment.
26. A topical ophthalmic formulation of embodiment 24 formulated as artificial tears.
27. A topical ophthalmic formulation of any of embodiments 24-26 wherein the NK1R agonist includes Substance P, a Substance P variant, GR73632, and/or GR64349.
28. A topical ophthalmic formulation of embodiments 27 wherein the NK1R agonist includes any of SEQ ID NOs: 2-6.
29. A topical ophthalmic formulation of any of embodiments 24-28 further including an IGF1 protein or peptide.
30. A topical ophthalmic formulation of embodiment 29 including SEQ ID NO: 8 or 9.

In particular embodiments, it may be beneficial to link an NK1R to methyl cellulose, for example:

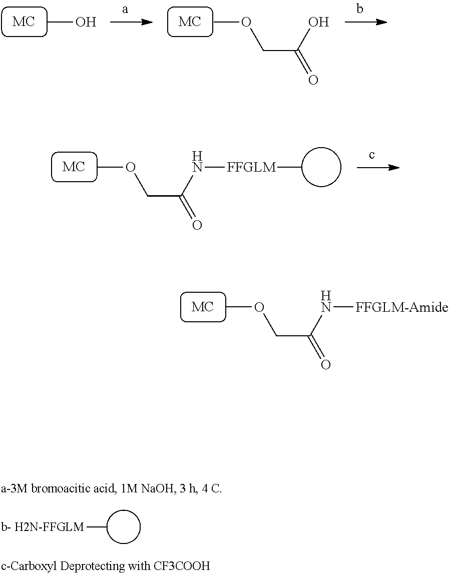

### Example 1

Introduction. Neurokinin-1 receptor (NK1R) is the highest affinity receptor for Substance P (SP), an eleven amino acid long neuropeptide. NK1R is expressed in corneal epithelial cells and SP-NK1R interaction induces chemokine responses in primary cultures of human corneal epithelial cells. Blocking NK1R signaling using NK1R antagonists ameliorates many pro-inflammatory conditions including airway and ocular inflammation in animal models. Therefore, NK1R serve as a promising target to control chronic inflammation. Currently, NK1R antagonists are approved to prevent nausea and vomiting associated with cancer chemotherapy in clinic. In addition to promoting inflammation, NK1R signaling is also reported to accelerate corneal epithelial wound healing in animal models [Nakamura et al., Invest Ophthalmol Vis Sci. 2003; 44(7):2937-40; Yang et al., Diabetes. 2014; 63(12):4262-74]. However, before the current disclosure, the role of functional NK1R in regulating ocular surface homeostasis/integrity under a steady-state condition was not known.

Naive corneas, for a long time, were considered an immune-privileged tissue partly because of the absence of immune cell types. However, recent studies challenged this notion by demonstrating the presence of a heterogeneous population of CD11c+ dendritic cells (DCs) in the corneal epithelium as well as in the anterior stroma of the cornea. Morphologically, corneal epithelial dendritic cells show long dendritic processes that cover a significant area of non-inflamed cornea. The density of these dendritiform CD11c+ DCs decreases from the peripheral to the central region of the naive cornea. Moreover, the majority of dendritiform CD11c+ DCs is present in the basal layer of the corneal epithelium and express a moderate to low level of MHC class II molecules, but lack or express a very low level of CD11b molecule. A recent study showed a physical association between corneal nerve fibers and epithelial DCs but the functional significance of this association is not known. Leppin et al., Invest Ophthalmol Vis Sci. 2014; 55(6):3603-15. On the other hand, the CD11c+ DCs present near the limbal area of naive cornea express more CD11 b molecules and are considered conventional DCs (cDCs) that are derived from the monocytic lineage. Overall, these studies show the presence of a heterogeneous population of DCs in normal cornea, but little is known about the role of NK1R in regulating the homeostasis of DCs in naive cornea.

Corneal DCs play an important role in orchestrating both innate and adaptive immune responses to ocular herpes simplex virus-1 (HSV-1) infection. In a mouse model of ocular HSV-1 infection, corneal DCs are involved in promoting HSV-1 clearance by regulating the influx of natural killer cells and inflammatory monocytes in infected corneas. On the other hand, DCs infiltrating the corneas at early time-points after ocular HSV-1 infection capture viral antigens and migrate to the draining lymph nodes (DLNs). In the DLNs, these migratory DCs are involved in priming virus specific T cells. Buela et al., J Immunol. 2015; 194(1):379-87. The role of corneal DCs in promoting systemic dissemination of virus after ocular HSV-1 infection has also been shown by Hu et al., PLoS One. 2015; 10(9):e0137123. Collectively, these studies demonstrate the functional significance of corneal DCs in the pathogenesis of ocular HSV-1 infection. However, the consequences of altered homeostasis of DCs in conjunctiva on the development of herpes stromal keratitis (HSK) have not been clear.

In this Example, it was determined that eyes obtained from unmanipulated NK1R^{-/-} mice exhibited an atypical expression of E-cadherin adhesion and Z0-1 tight junction proteins in the outermost layer of the corneal epithelium and were associated with excessive sloughing of the apical corneal epithelial (ACE) cells. To compensate for the loss of ACE cells, an increased proliferation of basal corneal epithelial cells was determined in eyes from NK1R^{-/-} mice. The lack of functional NK1R also resulted in an altered homeostasis of corneal epithelial and conjunctival DCs. Upon ocular HSV-1 infection, cDCs infiltrating the infected corneas and migrating to DLNs were much higher in NK1R^{-/-} mice and caused an increased priming of virus specific IFN-γ secreting CD4 T cells. Later, the increased number of CD4 T cells infiltrating the inflamed corneas of NK1R^{-/-} mice triggered an early development of severe HSK.

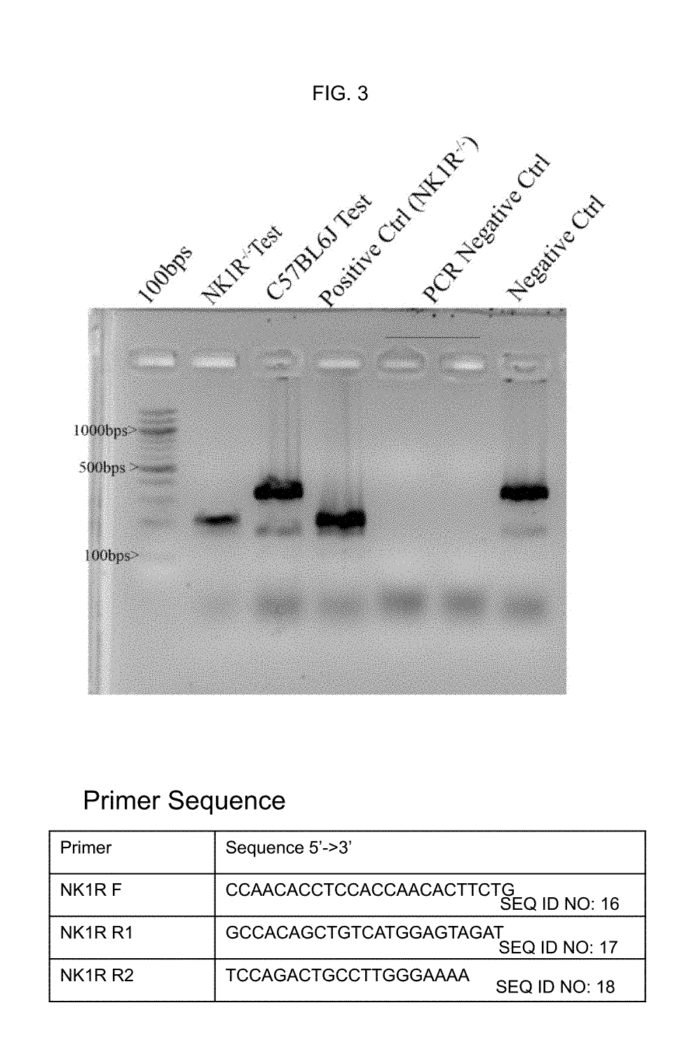

Materials and Methods. Mice. Eight to twelve weeks old female C57BL/6 (B6) mice were procured from the Jackson laboratory (Bar Harbor, Me.) and were housed in an Association for Assessment and Accreditation of Laboratory Animal Care (AAALAC) approved specific pathogen free animal facility at Wayne State University School of Medicine (WSUSOM). Special instructions were given to Jackson labs to ensure that the mice had no corneal opacity upon arrival. The NK1R (Tacr1) knockout mice pair was used as described in Bozic et al., Science. 1996; 273(5282):1722-5. The NK1R^{-/-} mice colony was bred and housed at the Division of Laboratory Animal Resources (DLAR) facility at WSUSOM. Functional ablation of NK1R gene in NK1R^{-/-} mice was confirmed using tail biopsy and performing polymerase chain reaction (PCR) followed by electrophoresis (FIG. 3). Animals were gender and age-matched for all experiments. All manipulations were performed in a type II biosafety cabinet. All experimental procedures were in complete agreement with the Association for Research in Vision and Ophthalmology resolution on the use of animals in research. In addition, all procedures were carried out in accordance with the rules and regulations of The Institutional Animal Care and Use Committee (IACUC) of Wayne State University.

Virus. The HSV-1 RE used in the current Example was propagated on a monolayer of Vero cells (American Type Culture Collection, Manassas, Va.; CCL81) as described previously in Gaddipati et al., J Immunol. 2015; 194(1):273-82. Pellets of infected Vero cells were suspended in 2 mL of 1× phosphate buffered solution (PBS) (Life Technologies, Grand Island, N.Y., USA; 14040-117) followed by three cycles of rapid freeze thaw with liquid nitrogen. Virus purified from the supernatant was titrated on Vero cells and stored in aliquots at −80° C. until used.

Corneal HSV-1 infection. To carry out ocular HSV-1 infection, mice were first anesthetized by intra-peritoneal injection of Ketamine (33 mg/Kg bodyweight)+Xylazine (20 mg/Kg bodyweight) in 0.2 ml PBS. The cornea was scarified with trephine (Fine Science Tools, Foster City, Calif.) while twisting three to four times over the corneal surface. 1×10⁴ plaque forming units (p.f.u) of HSV-1 virus were then topically applied with a pipette to the eye in 3 μL of 1×PBS followed by gentle massage of the eyelids.

Clinical Scoring of HSK. The eyes were examined on different days post-infection, while using a hand-held slit lamp biomicroscope (Kowa, Nagoya, Japan) to determine the extent of the corneal HSK and angiogenesis. A standard scale for corneal opacity, ranging from 0-5, was used as described in Gaddipati et al., J Immunol. 2015; 194(1):273-82. The neovascularization (NV) of the cornea was determined by measuring the centripetal growth of newly formed blood vessels in each quadrant of the cornea also as described in Gaddipati et al., J Immunol. 2015; 194(1):273-82.

Lymph nodes and corneal cell preparation for flow cytometry. HSV-1 infected C57BL/6 and NK1R^{-/-} mice were euthanized on day 7 post-infection. The draining lymph nodes (DLNs) were isolated and collected in RPMI+10FBS medium. A single cell suspension was prepared by triturating the lymphoid tissues with 3 ml syringe plungers followed by passage through a 70 μm cell strainer. Cells were counted using a hemocytometer under an inverted phase contrast microscope. The infected eyes from both groups of mice were enucleated and collected in ice-cold RPMI medium with antibiotics. Under a dissecting microscope, a radial incision was made at the limbal region and the cornea was separated from the underlying lens, iris, ciliary body and scleral tissue using curved fine forceps (Miltex surgical instruments, PA). The individual cornea was suspended into 250 μL of RPMI, and 20 μL of liberase TL (2.5 mg/mL) was added followed by incubation at 37° C. for 45 minutes on a tissue disruptor. At the end of the incubation period, the samples were triturated using 3 mL syringe plungers and passed through a 70 μm cell strainer followed by pelleting down the cells at 315×g for 8 minutes in a refrigerated centrifuge.

Cell surface staining. Cell surface staining was carried out as described in Gaddipati et al., J Immunol. 2015; 194(1):273-82. Briefly, the single cell suspension obtained from the individual cornea and DLN was washed with FACS buffer followed by blocking of Fe receptors and incubation with fluorochrome conjugated antibodies. The following antibodies were used for cell surface staining: Percp-cy5.5 conjugated-anti-CD4 (RM4-5), FITC conjugated anti-GrI (RB6-8C5), Alexa 647 conjugated anti-CD11c (N418). All of the antibodies were purchased from BD biosciences and BioLegend, San Diego, Calif. At the end of the cell surface staining, samples were acquired using a LSR II flow cytometer, and the data was analyzed using FlowJo software (Ashland, Oreg., USA. V8.8.7).

Intracellular cytokine and nuclear Ki-67 staining. Single cell suspensions of the lymph node were stimulated with 3 MOI (multiplicity of infection) of UV inactivated HSV-1. The virus was inactivated at 120,000 μJ/cm² for 1 min using XL-1000 UV cross linker (Spectronics Corporation). The cells were stimulated in a 96 well u-bottom plate for 16 hours at 37° C. in a C0₂ incubator. During the last four hours of incubation, 1 μl Brefeldin A (BD Golgi plug) was added to each well. At the end of the incubation period, cell surface staining was carried out followed by permeabilization of the cells with Cytofix/Cytoperm (BD biosciences). PE-conjugated anti-IFN-γ (XMG1.2) antibody was used to stain IFN-γ expressing CD4 T cells. The cells were washed in perm wash (BD Biosciences) buffer with the final washing given in FACS buffer. For nuclear Ki-67 staining, cells were stained using PE mouse anti-human Ki-67 kit (BD Pharmingen) as per the manufacturer's instruction. Samples were then acquired on LSR II flow cytometer and the data was analyzed using FlowJo software.

Mouse cytokine protein array analysis. Cytokine and chemokine levels in HSV-1 infected corneal tissues from B6 and NK1R^{-/-} mice were measured using Mouse Cytokine Array Panel A kit (R&D systems, Minneapolis, Minn.). Corneas dissected from infected eyes were transferred to 1×PBS with protease inhibitor cocktail (PIC) and kept at −80° C. Sonication of corneas was performed at 50% amplitude with a cycle of 15 second pulse followed by 1 minute of resting on ice. A total of six cycles was given to each cornea. Tissue lysates (sonicated samples) obtained were centrifuged at 4° C. at 15,000 rpm for 10 minutes. Supernatant was collected and the amount of protein in each sample was estimated using a BCA protein assay kit (Thermo Scientific). A total of 200 μg of protein from each group was used for cytokine and chemokine array analysis as per the manufacturer's instructions. Positive signals on the membranes were quantified using Image Studio Lite Version 4.0.

Corneal whole mount staining. Mice were euthanized and their eyes were enucleated with a pair of forceps. Eyeballs were immediately fixed in 4% paraformaldehyde for 30 min at room temperature. Corneal tissue was separated with a bard-parker #21 blade under a dissection microscope. Tissues were flattened with 6-8 partial cuts from the limbal to central cornea and stored in 30% sucrose solution overnight at 4° C. The next day, the corneas were permeabilized using 0.1% EDTA-0.01% Hyaluronidase type IV-S solution at 37° C. for 90 min, and immediately followed by blocking with 3% bovine serum albumin-1% Triton X100 solution for 90 min at room temperature. The corneal tissues were incubated with a primary antibody at appropriate dilution in a humidified chamber for 16 hours in a cold room (4° C.). The next day, the tissues were washed four times with 0.3% Triton X100-PBS solution (10 min each wash). If the primary antibody was unconjugated, a secondary antibody was added at appropriate dilution followed by overnight incubation in a cold room. Prior to acquisition, the corneal tissue was mounted with vectashield medium containing DAPI (H1200, Vector Laboratories, Burlingame, Calif., USA). Images were acquired using Leica TCS SP8 confocal microscope.

Cochet and Bonnet Esthesiometry. Corneal sensitivity was measured using a Cochet and Bonnet Esthesiometer attached with 12/100 mm nylon thread. This test was performed at 23-25° C. room temperature by touching the central cornea of B6 or NK1R^{-/-} mice. Numerical values were derived from a conversion table provided by the manufacturer (Luneau SAS).

Phenol red thread tear test. To measure the tear levels in the eyes of unmanipulated B6 and NK1R^{-/-} mice, pH indicator phenol red treated cotton threads were obtained from Zone-Quick (ZQ-1010, Showa Yakuhin Kako, Japan). The mice were restrained by holding the lose skin at the base of the neck without touching the facial skin or whiskers. The eyes were tested for tear level by placing the tip of the cotton thread on the bulbar conjunctiva of the lateral canthus for 30 seconds. A picture of the threads was taken and tear absorption was measured using a 10 cm scale.

Bromodeoxyuridine (BrdU) incorporation assay. For BrdU labeling of the corneal epithelium, 8 to 12-week old female B6 and NK1R^{-/-} mice were given a single intra-peritoneal injection of BrdU (10 mg BrdU/ml saline; 0.2 ml/mouse). The mice were euthanized on the next day after 18 hours and their eyes were collected to snap freeze in OCT media. Longitudinal sections were cut at 8 μm thickness, and mounted on positively charged slides (Thermo Shandon Limited, UK). The sections were air dried overnight at room temperature and fixed in 4% buffered paraformaldehyde for 15 min followed by 3 washings with 1×PBS. For antigen retrieval, sections were immersed in 2N HCl in a covered Coplin jar for 30 minutes at room temperature. The acid was neutralized with 0.1M Borate buffer (pH-9.0) for 5 minutes with two buffer changes. The slides were blocked with 3% BSA in 1×PBS and incubated with a primary antibody (Abeam, Mass., USA, BIM/75, ab6326) at 1:100 dilutions in blocking buffer for 18 hrs at 4° C. The next day, the slides were washed three times and incubated with Alexa-488 conjugated anti-Rat antibody (Molecular Probes) at 1:200 dilutions overnight at 4° C. The slides were washed and mounted with vectashield medium containing DAPI prior to acquisition.

Statistical Analysis. Statistical analysis was carried out using Graph Pad Prism software (San Diego, Calif.). All p values were calculated using an unpaired, two-tailed Student's t test. P<0.05 was considered statistically significant. The results are displayed as the mean.

Results. Atypical expression of E-cadherin adhesion and Z0-1 tight junction proteins in the corneal epithelium of NK1R^{-/-} mice.

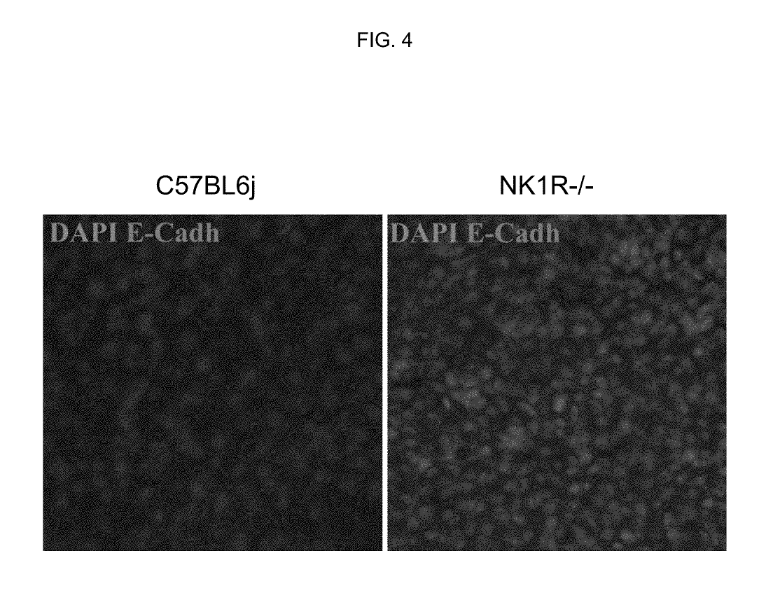

No phenotypic differences in the appearance of the eyes were noted when comparing C57BL/6 (B6) and NK1R^{-/-} mice (FIG. 1A). However, hematoxylin and eosin staining of paraffin-embedded eye sections from NK1R^{-/-} mice, but not B6 mice, showed corneal stromal edema, corneal epithelial hyperplasia, and irregular corneal epithelial surface (FIG. 1B). Additionally, a significant increase in numbers of hematoxylin stained nuclei was seen in the corneal epithelium of NK1R^{-/-} over B6 mice (FIG. 1C), suggesting an increased corneal epithelial proliferation in NK1R^{-/-} mice. Increased epithelial cell density and decreased epithelial cell size were also evident in the corneal flat mounts from NK1R^{-/-} mice (FIG. 4). Short-term BrdU incorporation assay confirmed an increased labeling of corneal epithelial cells in NK1R^{-/-} over B6 mice (FIG. 1D). One possible explanation for an increased epithelial proliferation in NK1R^{-/-} mice is to compensate for the excessive loss of cells from the outermost layer of the corneal epithelium. Exfoliation or sloughing of the apical corneal epithelial (ACE) cells is regulated by many factors including adhesion and tight junction proteins. Therefore, the expression patterns of E-cadherin adhesion and Z0-1 tight junction proteins in the outermost layer of the corneal epithelium were analyzed using corneal whole mount immunofluorescence staining. The results clearly showed the loss of E-cadherin and Z0-1 staining at selective areas in the limbal and central cornea of NK1R^{-/-} but not C57BL/6 mice (FIGS. 1E, 1F and 1G). In addition, the staining pattern of Z0-1 in the corneal epithelium of C57BL/6 mice was more complex than in NK1R^{-/-} mice. The corneal whole mount images clearly showed the areas with excessive loss of ACE cells (denoted by the * sign) in NK1R^{-/-} mice over wild type control B6 mice. Together, the results showed an atypical staining pattern of E-cadherin adhesion and Z0-1 tight junction proteins in the outermost layer of the corneal epithelium in association with an excessive sloughing of the ACE cells in NK1R^{-/-} mice.

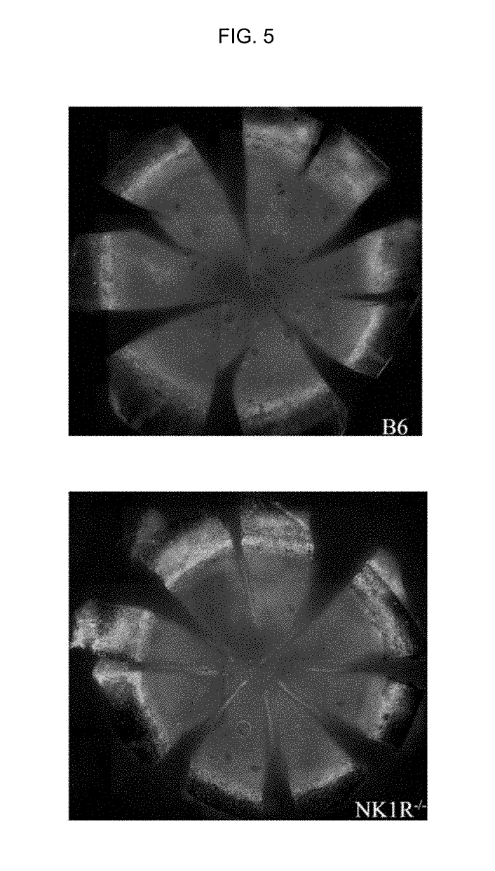

Loss of NK1R reduces the density of corneal epithelial DCs but dramatically increases the number of cDCs in bulbar conjunctiva and near the limbal area of cornea. The normal corneal epithelium is populated with CD11c+ DCs, which are in close association with surrounding epithelial cells. Resident corneal DCs promote the re-epithelialization of corneal wounds. Gao et al., Am J Pathol. 2011; 179(5):2243-53. Thus, an alteration in the homeostasis of corneal epithelium might affect the homeostasis of corneal DCs. Therefore, the density of corneal DCs was next compared by carrying out the immunofluorescence staining of CD11c+ cells in the corneal whole mounts from unmanipulated B6 and NK1R^{-/-} mice. As is shown in FIG. 2A, the resident corneal epithelial DCs present in the peripheral and central areas of NK1R^{-/-} mice corneas were almost half in number when compared to DCs in the corneas from B6 mice. Surprisingly, a more than two-fold increase in the numbers of CD11c+ cells was found near the limbal region of corneas from NK1R^{-/-} mice, when compared to the limbal areas of B6 mice corneas (FIG. 2B). The results also showed higher numbers of CD11c+ DCs in bulbar conjunctival tissue associated with flat mounts of whole corneas from NK1R^{-/-} over B6 mice (FIG. 5). Almost all of the CD11c+ cells near the limbal area were co-stained with CD11b molecule, suggesting that they were myeloid or conventional dendritic cells (cDCs) (FIG. 2C). Moreover, z-scans of the corneal whole mount near the limbal area showed that these cDCs were localized in both corneal epithelium and anterior stroma, whereas CD11b+CD11c-macrophages were deep seated in the stroma of the corneas from both groups of mice. Together, the results showed an altered homeostasis of conjunctival and corneal DCs in unmanipulated NK1R^{-/-} mice.

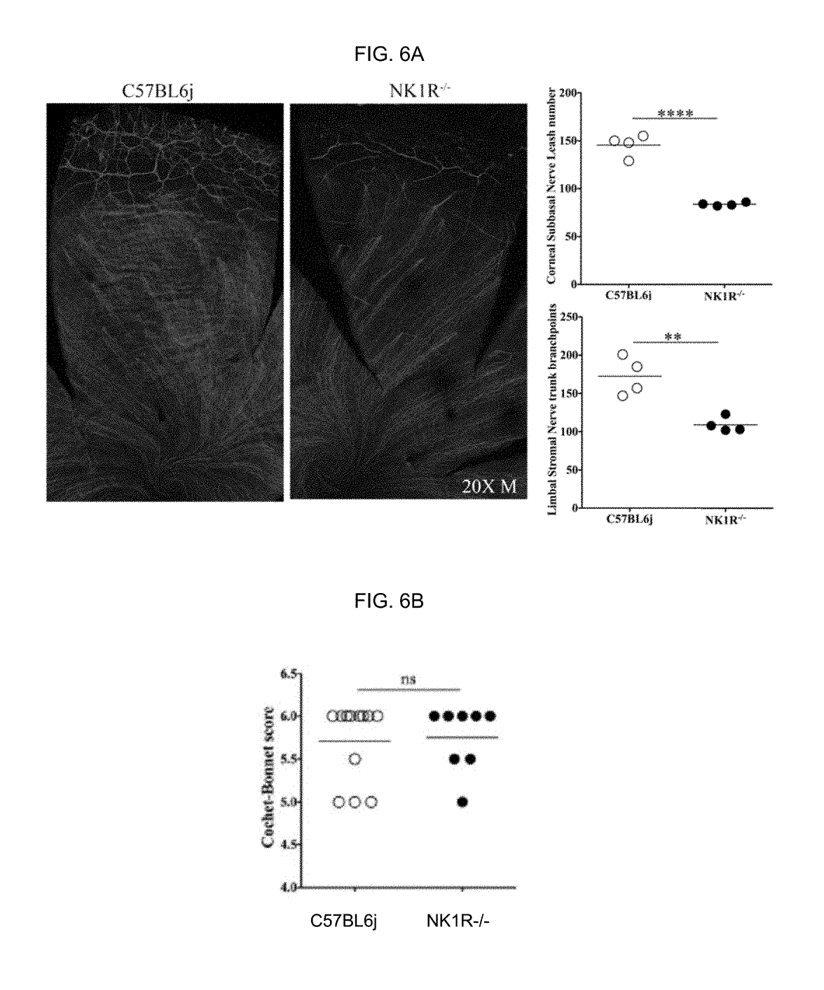

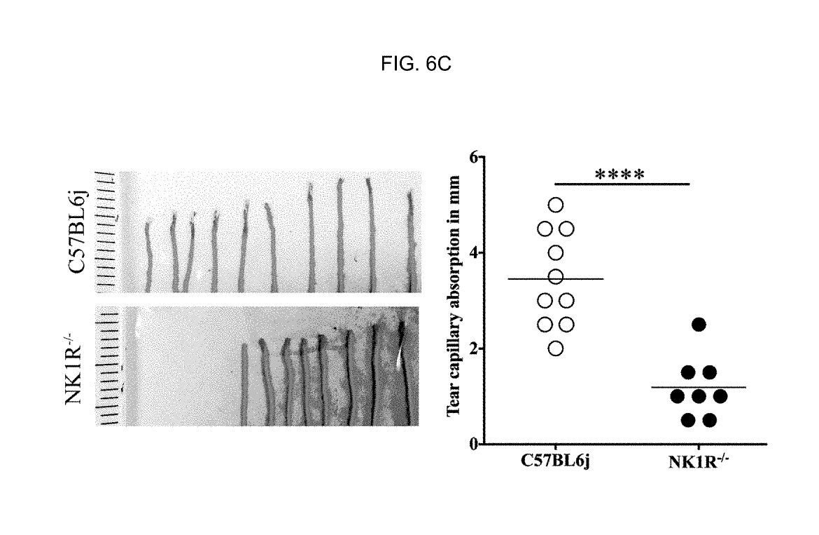

Reduced epithelial nerve density and stromal nerve trunk branching in the corneas from NK1R^{-/-} mice. The corneal epithelium is a highly innervated tissue and the corneal nerves are reported in close association with corneal epithelial cells and dendritic cells. Leppin et al., Invest Ophthalmol Vis Sci. 2014; 55(6):3603-15; Kubilus & Linsenmayer, Invest Ophthalmol Vis Sci. 2010; 51(2):782-9. It was next ascertained if altered homeostasis of corneal epithelial and dendritic cells influences the density of corneal nerves. The corneal whole mount staining for class III-tubulin, using Tuj-1 antibody, showed a significant decrease in the number of corneal subbasal nerve leashes in the peripheral region of the corneas from NK1R^{-/-} mice (FIG. 6A). Additionally, the number of corneal stromal nerve trunk branch points near the limbal area was also significantly reduced in NK1R^{-/-} over B6 mice (FIG. 6A). The corneal nerve density was similar in the central corneal region when comparing both groups of mice (data not shown). Moreover, no significant differences (p>0.05) in corneal sensation were determined in the central corneal region of the eyes from both groups of mice, when using the Cochet-Bonnet Esthesiometer (FIG. 6B). Lastly, the phenol red thread tear test was carried out to evaluate the volume of unstimulated tears in the eyes from NK1R^{-/-} and B6 mice. The results showed a statistically significant decrease (p<0.0001) in the volume of unstimulated tears in the eyes from NK1R^{-/-} over B6 mice (FIG. 6C).

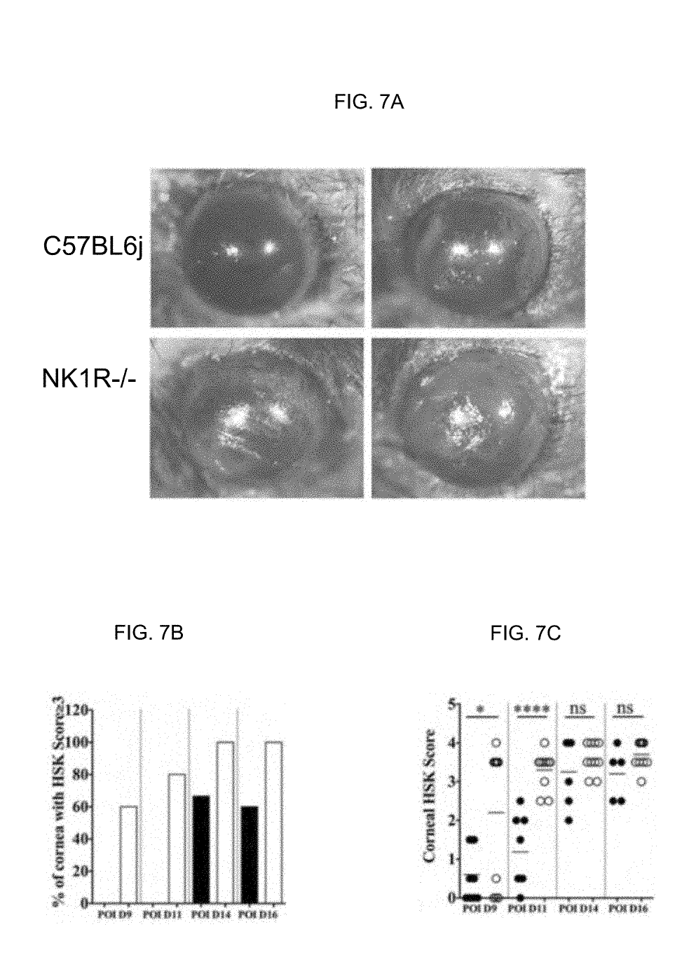

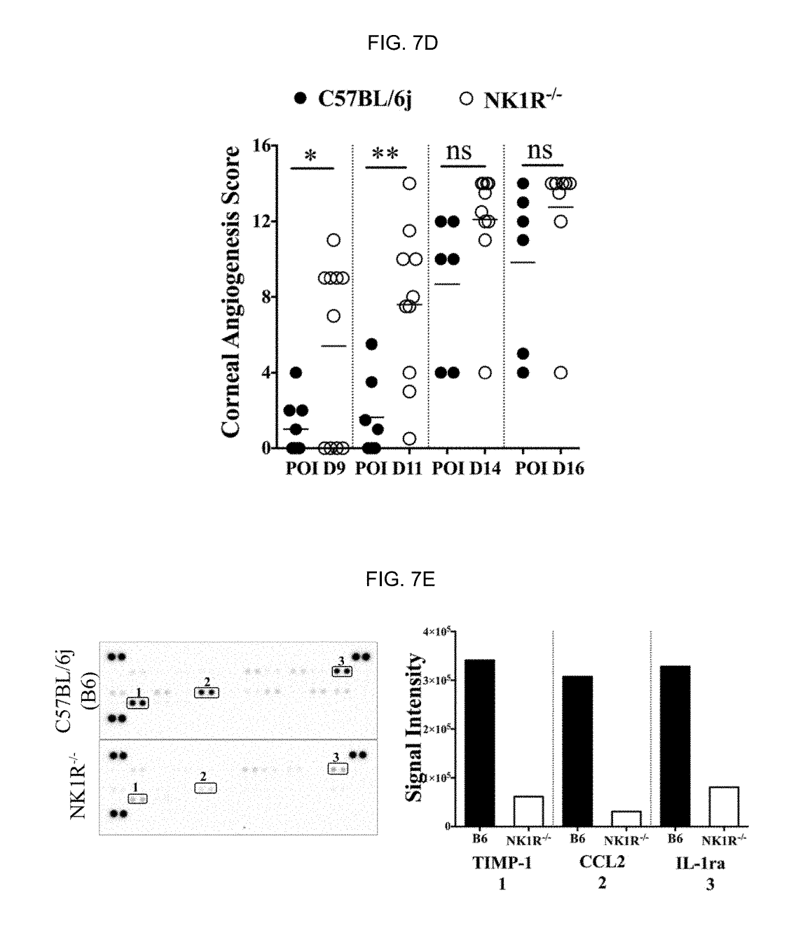

Ocular HSV-1 infection causes an early development of severe herpes stromal keratitis lesions in NK1R^{-/-} mice. NK1R signaling promotes inflammation after ocular microbial infection, and the use of NK1R antagonists reduces microbe induced ocular inflammation. Hazlett et al., Invest Ophthalmol Vis Sci. 2007; 48(2):797-807; Twardy et al., Invest Ophthalmol Vis Sci. 2011; 52(12):8604-13. Therefore, the development of herpes stromal keratitis (HSK) in B6 and NK1R^{-/-} mice after ocular HSV-1 infection were next compared. The corneas from both groups of mice were infected with HSV-1 as described in the methods section. Opacity and neovascularization of infected corneas from both groups of mice were scored using a hand-held slit lamp microscope on day 9, 11, 14, and 16 post infection. Unexpectedly, the results showed significantly increased corneal opacity and angiogenesis on day 9 and 11 post-infection in HSV-1 infected corneas from NK1R^{-/-} mice in comparison to the infected group of B6 mice (FIGS. 7A, 7C and 7D). The incidence of corneal opacity and angiogenesis was also much higher in NK1R^{-/-} mice when analyzed on day 9 and 11 post-infection (FIG. 7B). By day 14 and 16 post-infection, the extent of corneal opacity and angiogenesis was severe in the eyes from both groups of mice. It was next ascertained whether the presence or absence of functional NK1R regulated the level of cytokines in HSV-1 infected corneas during the development of HSK lesions. Infected corneas obtained from both groups of mice on day 9 post-infection were sonicated as described in the methods section. The amount of cytokines in the corneal lysates from both groups of mice was measured using a Mouse Cytokine Array assay kit as per the manufacturer's instructions. The results demonstrated that the infected corneal lysates from NK1R^{-/-} mice exhibited strikingly reduced levels of a number of cytokines including tissue inhibitor of metalloproteinase (TIMP-1), chemokine ligand 2 (CCL2) and interleukin-1 receptor antagonist (IL-1ra) (FIG. 7E).

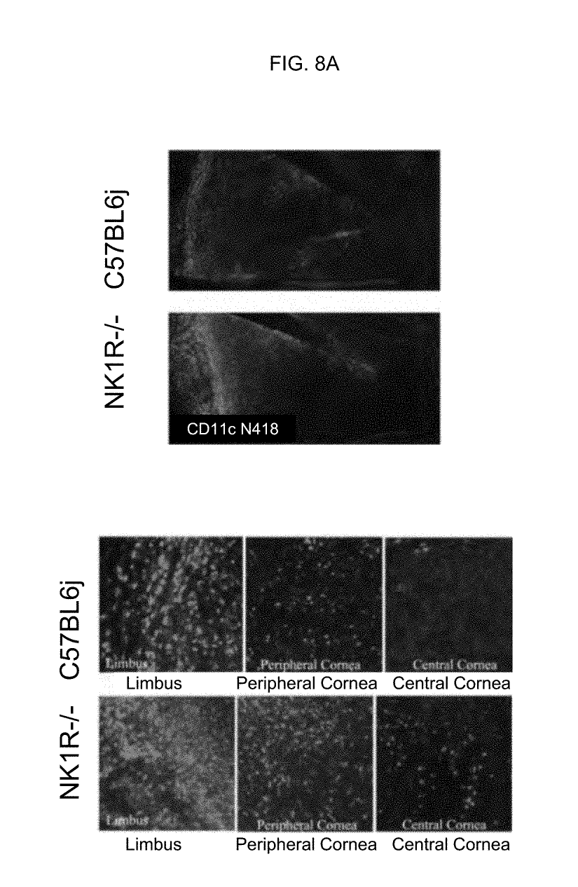

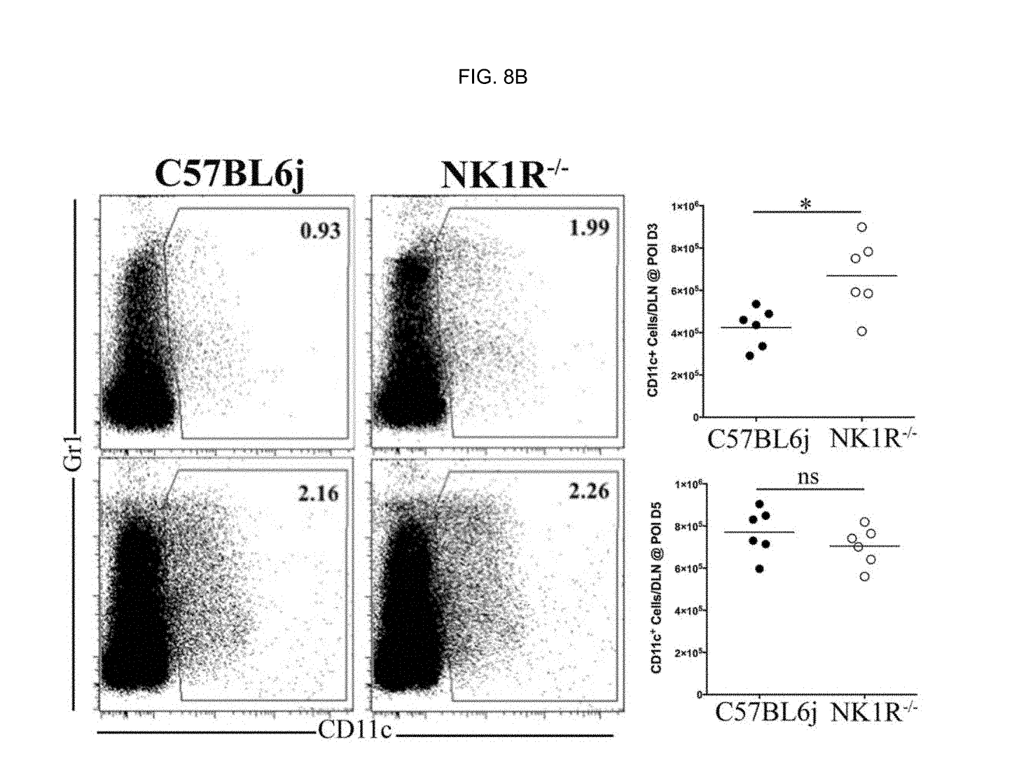

Increased numbers of DCs in corneas and draining lymph nodes of NK1R^{-/-} mice at early time point after corneal HSV-1 infection. The underlying cause for the early development of HSK in NK1R^{-/-} mice was ascertained next. DCs play an important role in the development of HSK. In light of the observation demonstrating the massive accumulation of cDCs near the corneal limbal area of unmanipulated NK1R^{-/-} mice, the influx of DCs in the peripheral, paracentral and central region of HSV-1 infected corneas of B6 and NK1R^{-/-} mice at early time-points post-infection was investigated. As is shown in the corneal whole mount staining of CD11c+ cells, NK1R^{-/-} mice exhibited an increased influx of DCs in all three regions of infected corneas as stated above on day 2 post-infection (FIG. 8A). The infiltrating DCs can pick up the viral antigens and migrate to the draining lymph nodes to prime virus specific T cells. Buela et al., J Immunol. 2015; 194(1):379-87. Therefore, the number of CD11c+ cells in draining lymph nodes of HSV-1 infected B6 and NK1R^{-/-} mice at early time-points post-infection were next ascertained. The results showed a significant increase in the absolute number of CD11c+ cells in the draining lymph nodes of NK1R^{-/-} over B6 mice on day 3, but not day 5 post ocular infection (FIG. 8B). Taken together, the results showed that in response to ocular HSV-1 infection, the numbers of dendritic cells infiltrating the infected corneas and then migrating to draining lymph nodes were higher in NK1R^{-/-} mice, when measured at early time points post-infection and thereby, suggested an increased priming of virus specific T cells in NK1R^{-/-} mice.

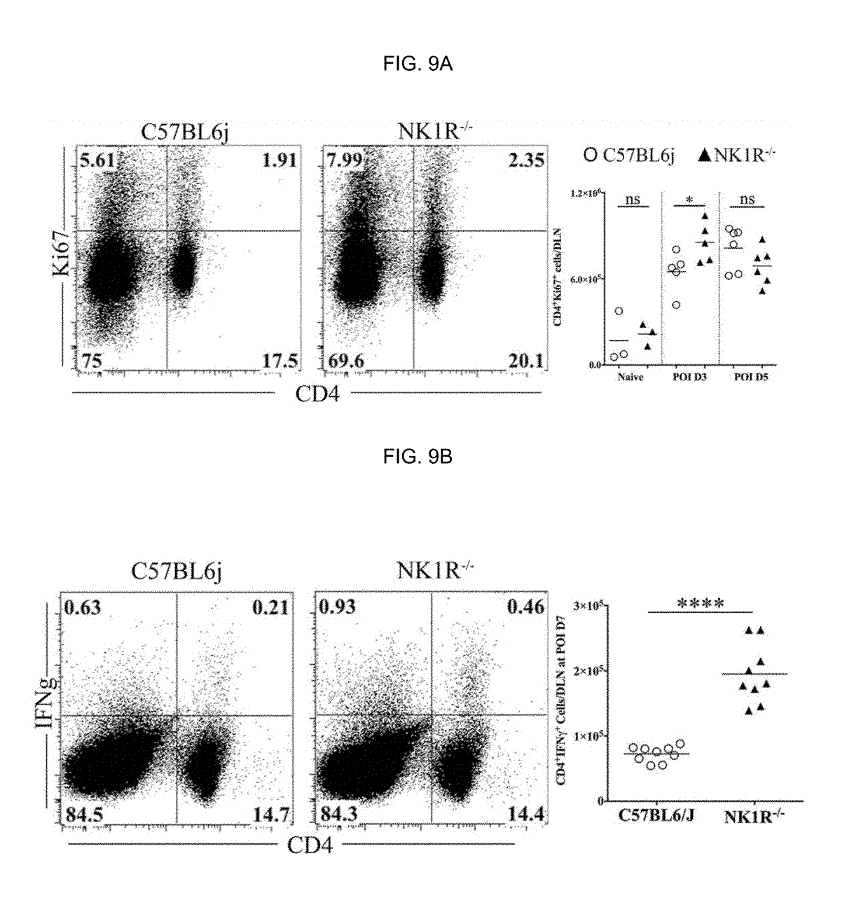

Increased expansion of CD4 T cells and IFN-γ secreting virus specific CD4 T cells in NK1R^{-/-} mice. After ocular HSV-1 infection, DCs migrating from infected corneas to DLNs are primarily responsible for expansion of CD4 T cells in DLNs. Buela et al., J Immunol. 2015; 194(1):379-87. Therefore, whether the increased number of DCs seen in the draining lymph nodes (DLNs) of HSV-1 infected NK1R^{-/-} mice at an early time-point (day 3) post-infection were functionally involved in enhancing the expansion and differentiation of CD4 T cells was ascertained next. The expansion of CD4 T cells in DLNs of infected B6 and NK1R^{-/-} mice was compared by measuring the expression of Ki-67 nuclear antigen on day 3 and 5 post-infection using flow cytometry. The results showed a significant increase in the proliferation of CD4 T cells in DLNs of NK1R^{-/-} mice on day 3, but not day 5 post-infection (FIG. 9A). To address the involvement of the increased population of DCs in differentiation of virus-specific CD4 T cells towards the Th1 subtype, DLNs from both groups of infected mice were homogenized to prepare a single cell suspension on day 7 post-infection. As described in the methods section, single cell suspensions from both groups of mice were stimulated with UV-inactivated HSV-1 followed by intracellular cytokine staining to determine the number of IFN-γ secreting CD4 T cells. As shown in FIG. 9B, a two-fold increase in the numbers of IFN-γ secreting virus specific CD4 T cells were detected in DLNs of infected NK1R^{-/-} mice when compared to infected B6 mice.

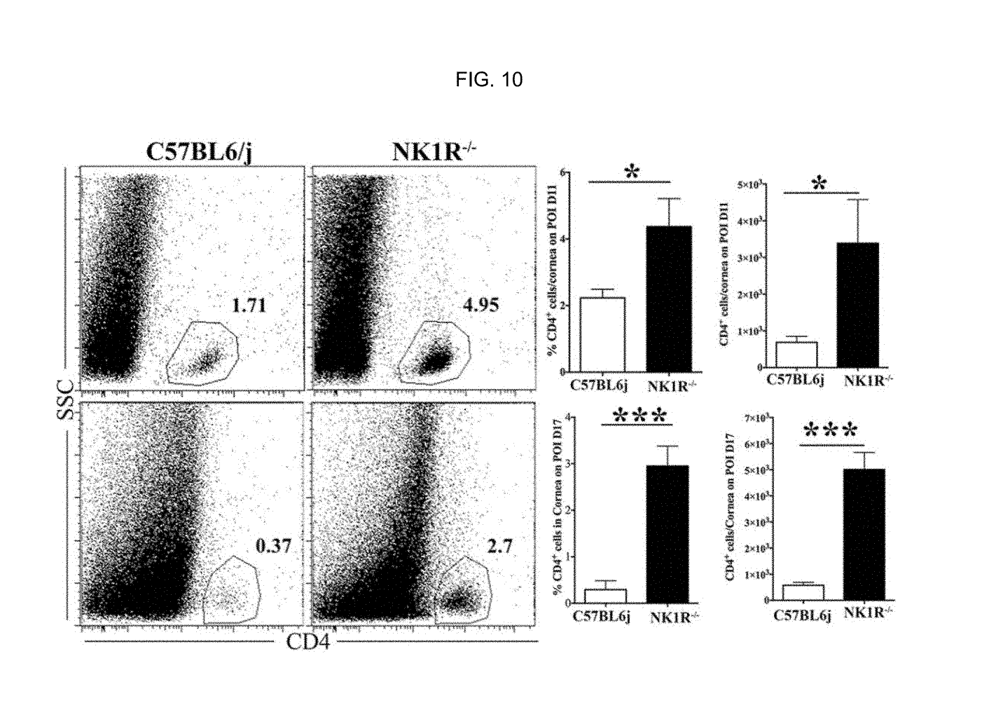

Increased influx of CD4 T cells into the inflamed corneas of infected NK1R^{-/-} mice. CD4 T cells play a pivotal role in the development of HSK. Therefore, the influx of CD4 T cells in inflamed corneas of HSV-1 infected NK1R^{-/-} and B6 mice was ascertained next. Single cell suspensions of individual corneas obtained from B6 and NK1R^{-/-} mice on day 11 and 17 post-infection were stained for CD4 T cells, and samples were acquired on a BD LSR II flow cytometer. The results showed an increased frequency and absolute number of CD4 T cells in infected corneas of NK1R^{-/-} mice on day 11 and 17 post-infection (FIG. 10). Together, the results suggest that DCs mediate increased expansion and Th1 differentiation of CD4 T cells in DLNs and play a key role in increased influx of helper T cells in inflamed corneas of NK1R^{-/-} mice and subsequently cause early development of HSK.

Discussion Corneal epithelial cells express NK1R, whereas the strongest affinity ligand of NK1R, Substance P, is present in sensory nerves innervating the corneal epithelium. SP-NK1R interaction promotes both sterile and microbial inflammation, and short-term blocking of NK1R signaling, using NK1R antagonists, ameliorates ocular surface inflammation in mouse models. Bignami et al., Invest Ophthalmol Vis Sci. 2014; 55(10):6783-94; Hazlett et al., Invest Ophthalmol Vis Sci. 2007; 48(2):797-807; Twardy et al., Invest Ophthalmol Vis Sci. 2011; 52(12):8604-13. In addition to promoting inflammation, NK1R signaling alone or in combination with insulin growth factor-1 (IGF-1) promotes corneal epithelial wound healing. Yang et al., Diabetes. 2014; 63(12):4262-74; Nagano et al., Invest Ophthalmol Vis Sci. 2003; 44(9):3810-5. Although the role of NK1R in inflammation and wound healing is well described, the contribution of NK1R in homeostasis of ocular surface under steady state (e.g., normal, healthy state) condition was not known before the current disclosure. In the current Example, while using NK1R^{-/-} mice, the role of NK1R in maintaining the homeostasis/integrity of corneal epithelial and ocular surface dendritic cells under steady state condition was characterized. The results also showed that eyes lacking functional NK1R, upon ocular HSV-1 infection, experience an early development and increased incidence of HSK.

The adult corneal epithelium is continuously renewed under steady state conditions. During the process of continuous renewal, the apical corneal epithelial (ACE) cells exfoliate or slough off at a regular basis and are replaced by the differentiating basal epithelial cells, which move up from the underlying layers. The corneal epithelial cells at the apical surface form tight junctions (TJs) and serve as a barrier to protect the ocular surface from microbial infections. In addition, the adherens junction (AJ) proteins in the corneal epithelium are involved in stabilization of cell-cell adhesion. (Hartsock & Nelson, Biochim Biophys Acta. 2008; 1778(3):660-9) and loss of E-cadherin affects proper formation of Z0-1 dependent tight junctions in skin epidermis. Tunggal et al., EMBO J. 2005; 24(6):1146-56. Thus, alteration in the formation of tight junction or adherens junction might affect the rate of exfoliation of ACE cells. The results support this notion, as it was determined that atypical expression of E-cadherin adhesion and Z0-1 tight junction proteins in the corneal epithelium of NK1R^{-/-} mice were associated with excessive sloughing of the ACE cells. Furthermore, the altered expression of E-cadherin adhesion and Z0-1 tight junction protein noted in the corneal epithelium of NK1R^{-/-} mice, is in support with previous findings showing SP mediated regulation of E-cadherin and Z0-1 protein expression in cultured human corneal epithelial cells. Ko et al., FEBS Lett. 2009; 583(12):2148-53; Araki-Sasaki et al., J Cell Physiol. 2000; 182(2):189-95. To maintain the corneal epithelium homeostasis, excessive loss of ACE cells should be compensated by hyper-proliferation of basal epithelial cells as determined in the corneas from NK1R^{-/-} mice (FIGS. 1A-1G). Additionally, rapid exfoliation of ACE cells may also impact the innervation of corneal nerves, as the latter forms complex structures with ACE cells. Kubilus & Linsenmayer, Invest Ophthalmol Vis Sci. 2010; 51(2):782-9. Due to excessive loss of ACE cells, the ends of nerve fibers innervating the corneal epithelium will not possibly make an association with corneal epithelial cells and may retract from the corneal epithelium, resulting in the lesser number of nerve leashes as seen in the results. Furthermore, the retraction of nerve fibers from the selective places in the corneal epithelium may also reduce the branching of stromal nerve trunks present near the limbal area of the cornea as depicted in the results. In fact, in human and B6 mouse models of HSK, decreased density of corneal nerves was shown to be associated with a reduced number of nerve trunks and total nerve branches. Hamrah et al., Ophthalmology. 2010; 117(10):1930-6; Chucair-Elliott et al., Invest Ophthalmol Vis Sci. 2015; 56(2):1097-107. In addition to its role in forming associations with corneal nerve endings, ACE cells also play an important role in holding tear film over the ocular surface. Thus, the reduced volume of basal tears measured in the eyes of NK1R^{-/-} mice is due to rapid exfoliation of ACE cells from the ocular surface of NK1R^{-/-} mice. Together, the findings document a novel role of NK1R in regulating the exfoliation of ACE cells in normal corneas under steady-state conditions.

It is widely accepted that the corneal epithelium is populated with CD11c+ cells with long dendritic processes, which cover a significant area of non-inflamed cornea. The intraepithelial DCs that largely reside in the basal layer of the corneal epithelium express langerin molecule and can be referred to as Langerhans cells (LCs). The density of intraepithelial DCs is much higher in peripheral and paracentral regions compared to central regions of uninfected corneas. These DCs and their dendritic processes are in close association with corneal epithelial cells, and loss of corneal DCs delays corneal epithelial wound healing in a mouse model of corneal epithelial injury. Gao et al., Am J Pathol. 2011; 179(5):2243-53. However, the nature of interaction between epithelial cells and resident DCs under steady state condition in corneal epithelium is not clear. On the other hand, in normal squamous epidermis, E-cadherin adhesion protein mediates the interaction between epidermal keratinocytes and Langerhans cells (LCs), and this interaction is required for retention of LCs in the skin. In fact, aberrant expression of E-cadherin in human papillomavirus infected cervical tissue was associated with a significant reduction in Langerhans cells. Leong et al., Br J Dermatol. 2010; 163(6): 1253-63. Therefore, atypical expression of E-cadherin noted in the corneal epithelium of NK1R^{-/-} mice is the most likely cause of the significant reduction in the number of corneal epithelial DCs noted in these mice. The results suggest that E-cadherin interactions play an important role in retention of corneal epithelial DCs as determined for LCs in the skin epidermis.

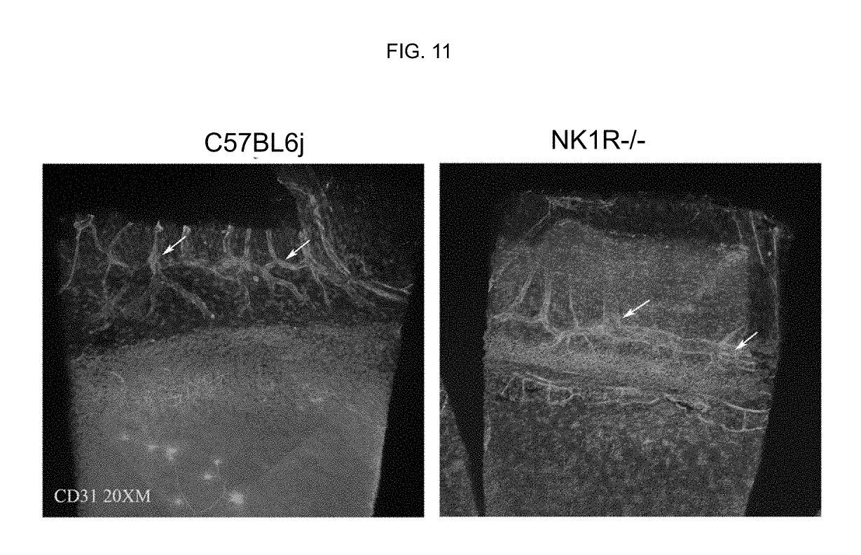

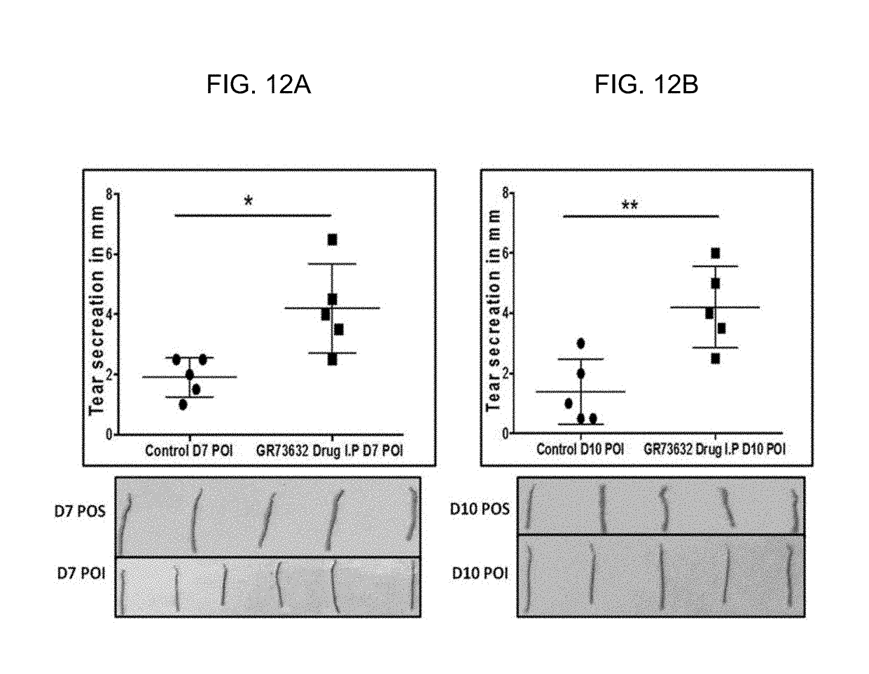

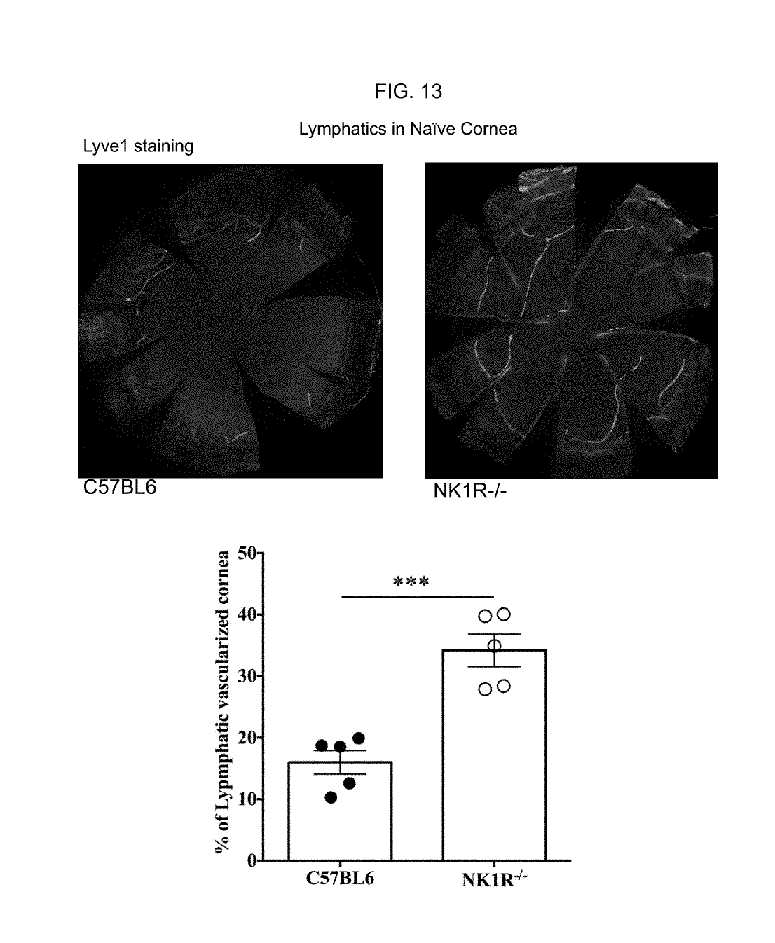

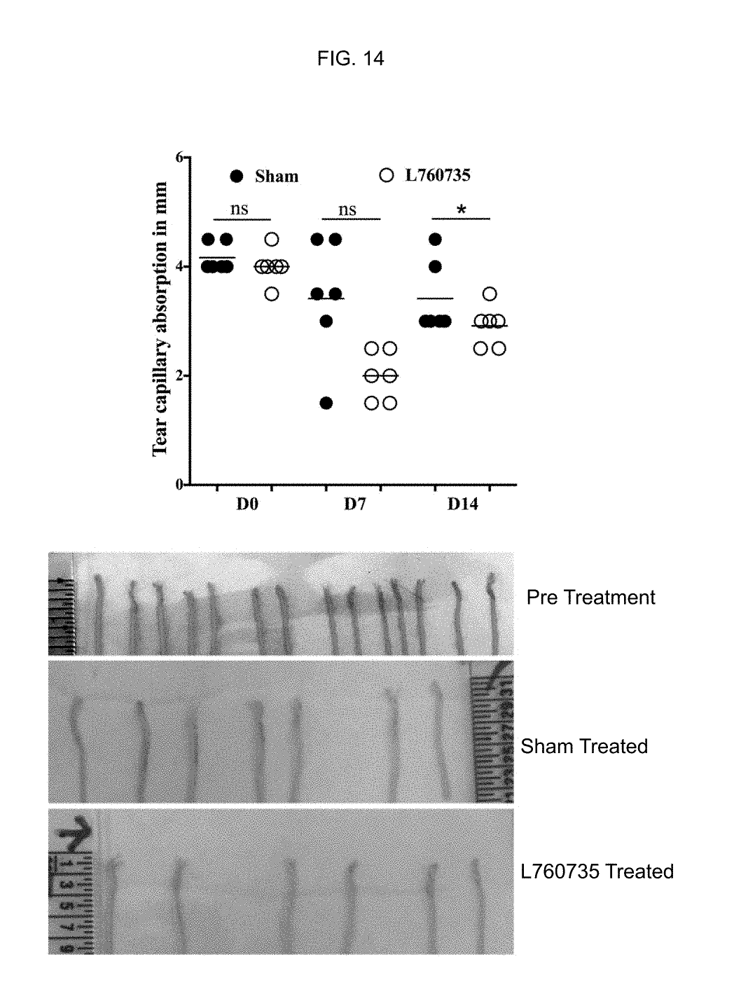

In addition to intraepithelial DCs, the normal cornea is also populated with cDCs in the anterior stroma. Recently, these cells were shown to express the langerin (c-type lectin) molecule as reported in case of dermal DCs that are present in the skin. Hattori et al., Invest Ophthalmol Vis Sci. 2011; 52(7):4598-604; Ginhoux et al., J Exp Med. 2007; 204(13):3133-46. Dermal DCs traffic to the skin dermis from the peripheral blood under steady state conditions. Ginhoux et al., J Exp Med. 2007; 204(13):3133-46. Similarly, it is possible that cDCs in the corneal limbal area, under steady state, are also recruited from the blood after extravasation from blood vessels present near the limbal area of normal corneas. The massive accumulation of cDCs noted in bulbar conjunctiva and near the corneal limbal area of NK1R^{-/-} mice could either be due to enhanced extravasation of these cells from conjunctival and limbal blood vessels or because of the exceeding number of cDCs in the peripheral circulation of NK1R^{-/-} mice. The results showed more vasodilation of feeder vessels near the limbal area of mice when compared with B6 mouse normal corneas (FIG. 11). No significant difference in the number of cDCs was determined in the peripheral blood of B6 and NK1R^{-/-} mice under steady state condition (data not shown). Vasodilation of blood vessels along with enhanced expression of selectins on vascular endothelial cells are known to cause an increased infiltration of leukocytes from the peripheral blood into the skin tissue. Ginhoux et al., J Exp Med. 2007; 204(13):3133-46. At present, the possible cause of an increased vasodilation depicted in the feeder blood vessels near the limbal area of NK1R^{-/-} mice is not clear. However, it was suspected that it may be due to SP interacting with other tachykinin receptors like NK2R or NK3R expressed on vascular endothelial cells in the absence of NK1R, as SP is known to promote vasodilation. These weaker affinity interactions could be longer lasting in comparison to the high-affinity SP-NK1R interaction which results in the internalization of SP and NK1R, thereby leading to desensitization.

DCs are reported to ferry microbial antigens from the mucosal tissues to the draining lymph nodes and prime antigen specific T cells to induce an adaptive immune response. In a recent study, infiltrating DCs, but not resident epithelial dendritic cells, were shown to cause expansion of CD4 T cells in DLNs after ocular HSV-1 infection in a mouse model. Buela et al., J Immunol. 2015; 194(1):379-87. The results are in agreement with this Example as the increased population of DCs noted near the corneal limbal area of NK1R^{-/-} mice infiltrate HSV-1 infected corneas after ocular HSV-1 infection, migrate to draining lymph nodes in large numbers and cause increased expansion and Th1 differentiation of CD4 T cells in comparison to infected B6 mice. The severity of HSK in a mouse model is determined by many factors, but CD4 T cells in HSV-1 infected corneas play a key role in the development of corneal pathology. The results showed a positive correlation between increased numbers of CD4 T cells in HSV-1 infected corneas and severity of HSK in NK1R^{-/-} mice.

Together, the findings characterized a novel role of NK1R in maintaining the homeostasis/integrity of the ocular surface under steady state conditions (Schematics; FIG. 15).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in the ability of an embodiment to reduce dry eye according to an experimental model described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of reducing dry eye and ocular infections in a subject having reduced N1KR activity comprising administering a therapeutically effective amount of a Neurokinin 1 Receptor (NK1R) agonist to the subject thereby reducing dry eye and ocular infections in the subject having reduced N1KR activity.

2. A method of claim 1, wherein the subject has reduced NK1R activity due to administration of an NK1R antagonist.

3. A method of claim 2, wherein the administration of the NK1R antagonist is to treat a condition.

4. A method of claim 3, wherein the condition is alcoholism, Sezary Syndrome, epidermolysis Bullosa, nausea, vomiting, post-traumatic stress disorder, or HIV infection.

5. A method of claim 1, wherein the administering is topical ocular administering.

6. A method of claim 1, wherein the NK1R agonist is Substance P or a Substance P variant selected from SEQ ID NOs: 2-6.

7. A method of claim 1, wherein the NK1R agonist is PEGylated.

8. A method of claim 1, further comprising administering an IGF1 protein or peptide.

9. A method of claim 1, wherein the administering leads to an increase in tear volume for the subject.
